SEQUENCE LISTING (18 sequences)

SEQ ID NO: 1 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnncagrtgcagctggtgcart

SEQ ID NO: 2 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnnsaggtccagctggtrcagt

SEQ ID NO: 3 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnncagrtcaccttgaaggagt

SEQ ID NO: 4 - DNA, 74 bp, synthetic construct
  source 1..74: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnnsaggtgcagctggtggag

SEQ ID NO: 5 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnncaggtgcagctacagcagt

SEQ ID NO: 6 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnncagstgcagctgcaggagt

SEQ ID NO: 7 - DNA, 75 bp, synthetic construct
  source 1..75: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnngargtgcagctggtgcagt

SEQ ID NO: 8 - DNA, 76 bp, synthetic construct
  source 1..76: mol_type = other DNA
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
aatgatacggcgaccaccgagatctacacgttgtaaaacgacggccagtnnnnnnncaggtacagctgcagcagtc

SEQ ID NO: 9 - DNA, 24 bp, synthetic construct
  source 1..24: mol_type = other DNA
caagcagaagacggcatacgagat

SEQ ID NO: 10 - DNA, 32 bp, synthetic construct
  source 1..32: mol_type = other DNA
agtcagtcagccgacsgatgggcccttggtgg

SEQ ID NO: 11 - DNA, 34 bp, synthetic construct
  source 1..34: mol_type = other DNA
agtcagtcagccggaattctcacaggagacgagg

SEQ ID NO: 12 - DNA, 38 bp, synthetic construct
  source 1..38: mol_type = other DNA
gaccaccgagatctacacgttgtaaaacgacggccagt

SEQ ID NO: 13 - DNA, 33 bp, synthetic construct
  source 1..33: mol_type = other DNA
acactctttccctacacgacgctcttccgatct

SEQ ID NO: 14 - DNA, 32 bp, synthetic construct
  source 1..32: mol_type = other DNA
ccaccaagggcccatcsgtcggctgactgact

SEQ ID NO: 15 - DNA, 37 bp, synthetic construct
  source 1..37: mol_type = other DNA
cggtctcggcattcctgctgaaccgctcttccgatct

SEQ ID NO: 16 - DNA, 34 bp, synthetic construct
  source 1..34: mol_type = other DNA
cctcgtctcctgtgagaattccggctgactgact

SEQ ID NO: 17 - DNA, 31 bp, synthetic construct
  source 1..31: mol_type = other DNA
acgagggggaaaagggttggggcggatgcac

SEQ ID NO: 18 - protein, 88 aa, synthetic construct
GAGLLKPSETLSLTCAVYGGSFSGYYWSWIRQPPGKGLEWIGEINHSGSTNYNPSLKSRVTISVDTSKNQFSLKLSSVTAADTAVYYC

(19) United States
(12) Patent Application Publication
(10) Pub. No.: US 2025/0333794 A1
(43) Pub. Date: Oct. 30, 2025

(54) HYPOGAMMAGLOBULINEMIA PATIENT SELECTION FOR IMMUNOGLOBULIN REPLACEMENT THERAPY

(71) Applicants: GigaGen, Inc., San Carlos, CA (US); c/o Albert-Ludwigs-Universität Freiburg, Freiburg (DE)

(72) Inventors: Adam Shultz ADLER, Belmont, CA (US); Yoong Wearn LIM, Pacifica, CA (US); Bodo GRIMBACHER, Freiburg (DE); Klaus WARNATZ, Freiburg (DE)

(21) Appl. No.: 19/264,586

(22) Filed: Jul. 9, 2025

Related U.S. Application Data
(63) Continuation of application No. PCT/US2024/011513, filed on Jan. 12, 2024.
(60) Provisional application No. 63/439,054, filed on Jan. 13, 2023.

(51) Int. Cl. C12Q 1/6883; G16B 40/00; G16H 50/20

(52) U.S. Cl. CPC C12Q 1/6883; G16B 40/00; G16H 50/20; C12Q 2600/106; C12Q 2600/156

(57) ABSTRACT

The present disclosure provides a method of selecting a hypogammaglobulinemia patient that needs an immunoglobulin replacement therapy (IgG-RT) by analyzing the patient's B cell repertoire. The method can be used before treatment of the patient with IgG-RT. Further provided herein include a diagnostic product providing information for the patient selection and the method of diagnosis.

### 1. CROSS-REFERENCE TO RELATED APPLICATIONS

This application is a continuation of International Application No. PCT/US2024/011513, filed Jan. 12, 2024, which claims the benefit of U.S. Provisional Application No. 63/439,054, filed Jan. 13, 2023, each of which is hereby incorporated in its entirety by reference.

### 2. SEQUENCE LISTING

The instant application contains a Sequence Listing which has been submitted electronically via Patent Center and is hereby incorporated by reference in its entirety. Said XML copy, created on Jan. 9, 2024, is named 54256WO_CRF_sequencelisting.xml, and is 39,316 bytes in size.

### 3. BACKGROUND OF THE INVENTION

Defense against infections is orchestrated by a complex immune system where every component has a task, and the quantitative or qualitative defect of a single component often contributes to a clinically apparent immunodeficiency. The most common form of inborn errors of immunity/primary immunodeficiency are antibody deficiencies, a phenotype which is mostly characterized by recurrent upper respiratory tract infections. Antibody deficiencies include agammaglobulinemia (no antibodies), hypogammaglobulinemia (not enough antibodies), IgG subclass deficiencies, and specific anti-PnPS (pneumococcal polysaccharide) deficiency, the latter presenting with recurrent pneumococcal infections.

The combination of serum IgG levels and infections susceptibility have been used to make the decision for or against providing IgG-RT, as the immunoglobulin replacement preparations do not contain significant amounts of IgM or IgA. Hence, IgG-RT is not indicated for the treatment of selective IgA deficiency. The reduction of an IgG titer to 4 g/L has been believed to be associated with an increased risk of infection, though some patients with almost normal IgG levels may still present pathological infection susceptibility. Conversely, some people with IgG levels of <4 g/L show no apparent infection susceptibility, potentially because their immune system can respond to each challenge with high quality acute naïve and memory IgG responses.

Accordingly, there is a clinical conundrum of why some patients with severe hypogammaglobulinemia have no infection susceptibility and thus do not need IgG-RT, while most of the patients with antibody deficiency need IgG-RT to stay healthy. There is a need to develop a reliable way to identify patients who requires immunoglobulin replacement therapy (IgG-RT).

### 4. SUMMARY OF THE INVENTION

Applicant tested whether the composition of the peripheral B cell receptor sequences and number/diversity of B cell clones provide an indication of why some patients with severe hypogammaglobulinemia have no infection susceptibility and thus do not need IgG-RT, while most of the patients with antibody deficiency need IgG-RT to stay healthy. Specifically, Applicant sequenced and analyzed the IgG and IgM heavy chain B cell receptor repertoires from PBMCs isolated from cohorts of patients with low serum IgG concentrations who did or did not require IgG-RT. The experimental data showed that patients who needed IgG-RT had more diverse IgG and IgM antibody repertoires, and their IgG sequences were significantly more similar to germline. This suggests that, although patients with low serum IgG concentrations who required IgG-RT had higher diverse repertoires, their antibody clones were less diverged from germline and thus might not be as optimal for targeting pathogens, causing infection susceptibility. Conversely, those with low serum IgG concentrations who did not need IgG-RT had lower diverse, yet more matured, antibody sequences, which might be better suited to targeting pathogens.

Based on the study, the present application provides a method of selecting a hypogammaglobulinemia patient for immunoglobulin replacement therapy (IgG-RT), comprising
(1) obtaining sequence information of at least 10,000 transcripts from the patient's sample comprising B cells, wherein each of the transcripts encodes a heavy chain of IgG or IgM or a portion thereof; and
(2) characterizing B cell receptor (BCR) repertoire of the patient by identifying antibody clones using the sequence information;
(3) analyzing the BCR repertoire by one or more selected from (a)-(f):
   (a) measuring the number or abundance of individual antibody clones in the BCR repertoire;
   (b) calculating a diversity index value of the antibody clones by measuring the number and abundance of individual antibody clones in the BCR repertoire;
   (c) selecting at least 10 but no more than 30 antibody clones that are most frequent in the BCR repertoire and calculating a total frequency of the 10 to 30 most frequent antibody clones;
   (d) determining a variable region gene (V region) usage frequency in the antibody clones, optionally wherein the V region gene is selected from the group consisting of IGHV4-30-2 heavy chain V region gene, IGHV4-30-4 heavy chain V region gene, IGHV3-23 gene, IGHV4-34 heavy chain V region gene, and IGHV4-31 heavy chain V region gene;
   (e) measuring a percent germline identity by comparing the V or J region in the BCR repertoire against a corresponding germline sequence; and
   (f) determining the somatic mutation frequency in different regions along the V region;
(4) selecting a patient for IgG-RT when one or more of (g)-(s) are satisfied:
   (g) the number of IgG clones in the BCR repertoire is greater than Tg, wherein Tg is at least 600;
   (h) the diversity index value of the IgM antibody clones is greater than Th, wherein Th is at least 250;
   (i) the total frequency of the most frequent 10 to 30 IgG antibody clones in the BCR repertoire is less than Ti, wherein Ti is at most 30%;
   (j) the total frequency of the most frequent 10 to 30 IgM antibody clones in the BCR repertoire is less than Tj, wherein Tj is at most 7%;
   (k) the frequency of IgG antibody clones with the IGHV4-30-2 heavy chain V region is less than Tk, wherein Tk is at most 0.3%;
   (l) the frequency of IgG antibody clones with the IGHV4-30-4 heavy chain V region is less than Tl, wherein Tl is at most 0.5%;
   (m) the frequency of IgG antibody clones with the IGHV3-23 heavy chain V region is greater than Tm, wherein Tm is at least 6%;
   (n) the frequency of IgG antibody clones with the IGHV4-34 heavy chain V region is greater than Tn, wherein Tn is at least 6%;
   (o) the frequency of IgM antibody clones with the IGHV4-31 heavy chain V region is less than To, wherein To is at most 0.2%;
   (p) IgG V gene average percent germline identity is greater than Tp, wherein Tp is at least 98%;
   (q) IgG J gene average percent germline identity is greater than Tq, wherein Tq is at least 98%;
   (r) the median somatic nucleotide mutation frequency in the FR1, CDR1, FR2, or CDR2 regions of IgG is lower than Tr, wherein Tr is at most 1; and
   (s) the median somatic nucleotide mutation frequency in the FR3 region of IgG is less than Ts, wherein Ts is at most 4;
(5) providing information related to whether or not the patient needs IgG-RT.

In some embodiments, in step (1), obtaining sequence information of at least 50,000 transcripts. In some embodiments, in step (1), obtaining sequence information of at least 100,000, 500,000, 1000,000 or 10 million transcripts.

In some embodiments, in step (4), the patient for IgG-RT is selected when (g) is satisfied. In some embodiments, in (g), Tg is 600, 650, 700, 750, 800, 850, 900, 950, 1,000, 1,050, or 1,100. In some embodiments, in (g), Tg is between 700 and 1,200, between 700 and 1,100, between 700 and 1,000, between 800 and 1,100, between 800 and 1,000, between 900 and 1,100, between 900 and 1,000, between 800 and 900, or between 700 and 800.

In some embodiments, in step (4), the patient for IgG-RT is selected when (h) is satisfied. In some embodiments, in (h), Th is 250, 300, 350, 400, 450, 500, 550, or 600. In some embodiments, in (h), Th is between 250 and 650, between 300 and 650, between 350 and 650, between 400 and 650, between 450 and 650, between 250 and 600, between 300 and 600, between 350 and 600, between 400 and 600, between 450 and 600, between 250 and 550, between 300 and 550, between 350 and 550, between 400 and 550, between 450 and 550, between 250 and 500, between 300 and 500, between 350 and 500, between 400 and 500, or between 450 and 500.

In some embodiments, in step (4), the patient for IgG-RT is selected when (i) is satisfied. In some embodiments, in (i), Ti is 30%, 25%, or 20%. In some embodiments, in (i), Ti is between 20% and 35%, between 20% and 30%, between 20% and 25% or between 25% and 35%, or between 25% and 30%.

In some embodiments, in step (4), the patient for IgG-RT is selected when (j) is satisfied. In some embodiments, in (j), Tj is 7%, 6.5%, 6%, 5.5%, 5%, 4.5%, or 4%. In some embodiments, in (j), Tj is between 4% and 7%, between 4% and 6%, between 4% and 5%, between 5% and 7%, between 5% and 6%, or between 6% and 7%.

In some embodiments, in step (4), the patient for IgG-RT is selected when (k) is satisfied. In some embodiments, in (k), Tk is 0.3%, 0.25%, 0.2%, or 0.15%. In some embodiments, in (k), Tk is between 0.15% and 0.3%, between 0.2% and 0.3%, between 0.25% and 0.3%, between 0.15% and 0.25%, between 0.15% and 0.2%, between 0.2% and 0.3%, or between 0.25% and 0.3%.

In some embodiments, in step (4), the patient for IgG-RT is selected when (l) is satisfied. In some embodiments, in (l), Tl is 0.5%, 0.45%, 0.4%, 0.35%, 0.3%, or 0.25%. In some embodiments, in (l), Tl is between 0.25% and 0.5%, between 0.25% and 0.45%, between 0.25% and 0.4%, between 0.25% and 0.35%, between 0.25% and 0.3%, between 0.3% and 0.5%, between 0.3% and 0.45%, between 0.3% and 0.4%, between 0.3% and 0.35%, between 0.35% and 0.45%, between 0.35% and 0.4%, between 0.4% and 0.5%, between 0.4% and 0.45%, or between 0.45% and 0.5%.

In some embodiments, in step (4), the patient for IgG-RT is selected when (m) is satisfied. In some embodiments, in (m), Tm is 6%, 7%, 8%, 9%, or 10%. In some embodiments, in (m), Tm is between 6% and 10%, between 6% and 9%, between 6% and 8%, between 6% and 7%, between 7% and 10%, between 7% and 8%, between 8% and 10%, between 8% and 9%, or between 9% and 10%.

In some embodiments, in step (4), the patient for IgG-RT is selected when (n) is satisfied. In some embodiments, in (n), Tn is 6%, 7%, 8%, 9%, or 10%. In some embodiments, in (n), Tn is between 6% and 10%, between 6% and 9%, between 6% and 8%, between 6% and 7%, between 7% and 10%, between 7% and 8%, between 8% and 10%, between 8% and 9%, or between 9% and 10%.

In some embodiments, in step (4), the patient for IgG-RT is selected when (o) is satisfied. In some embodiments, in (o), To is 0.2%, 0.15%, or 0.1%. In some embodiments, in (o), To is between 0.1% and 0.2%, between 0.1% and 0.15%, or between 0.15% and 0.2%.

In some embodiments, in step (4), the patient for IgG-RT is selected when (p) is satisfied. In some embodiments, in (p), Tp is 98%, 98.5% or 99%. In some embodiments, in (p), Tp is between 98% and 99%, between 98% and 98.5%, or between 98.5% and 99%.

In some embodiments, in step (4), the patient for IgG-RT is selected when (q) is satisfied. In some embodiments, in (q), Tq is 98%, 98.5% or 99%. In some embodiments, in (q), Tq is between 98% and 99%, between 98% and 98.5%, or between 98.5% and 99%.

In some embodiments, in step (4), the patient for IgG-RT is selected when (r) is satisfied. In some embodiments, in (r), Tr is 1, 0.8, or 0.6. In some embodiments, in (r), Tr is between 0.6 and 1, between 0.6 and 0.8, or between 0.8 and 1.

In some embodiments, in step (4), the patient for IgG-RT is selected when(s) is satisfied. In some embodiments, in(s), Ts is 4, 3.5, 3, 2.5, or 2. In some embodiments, in(s), Ts is between 2 and 4, between 2 and 3.5, between 2 and 3, between 2 and 2.5, between 2.5 and 4, between 2.5 and 3.5, between 2.5 and 3, between 3 and 4, between 3 and 3.5, or between 3.5 and 4.

In some embodiments, in step (3), one, two, three, four, five, or six analysis out of (a) to (f) are performed for analysis of BCR repertoire.

In some embodiments, in step (4), the patient for IgG-RT is selected when one, two, three, four, five, six, seven, eight, nine, ten, eleven, twelve, or thirteen criteria selected from (g) to (s) are satisfied.

In some embodiments, in step (4), the patient for IgG-RT is selected when at least two, at least three, at least four, at least five, at least six, at least seven, at least eight, at least nine, at least ten, at least eleven criteria, or at least twelve criteria selected from (g) to (s) are satisfied.

In some embodiments, the patient has been selected for having less than 5 g/L of serum IgG and more than 40/μL of peripheral B cells. In some embodiments, the patient has been selected for having less than 4.5 g/L of serum IgG and more than 35/μL of peripheral B cells. In some embodiments, the patient has been selected for having less than 4 g/L of serum IgG and more than 30/μL of peripheral B cells.

In some embodiments, the patient's sample comprises peripheral blood mononuclear cells (PBMCs). In some embodiments, the method further comprises the step of sequencing the at least 10,000 transcripts, thereby providing the sequence information. In some embodiments, the method further comprises the step of treating the patient with IgG-RT when the patient is selected for IgG-RT.

In another aspect, the present disclosure provides a method of treating a hypogammaglobulinemia patient, comprising administering immunoglobulin replacement therapy (IgG-RT) to the patient, wherein the patient has been selected for IgG-RT using the method disclosed herein. In some embodiments, the method further comprises selecting the patient for IgG-RT using the method disclosed herein.

In yet another aspect, the present disclosure provides a diagnostic product for selecting a hypogammaglobulinemia patient for treatment with immunoglobulin replacement therapy (IgG-RT), wherein the diagnostic product is stored on a non-transitory computer readable medium and is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) characterizing B cell receptor (BCR) repertoire of each of the patients by identifying antibody clones based on the sequence information;
(3) obtaining a training dataset including a plurality of training examples, wherein each training example corresponds to a BCR repertoire of an individual patient and comprises;
   (a) one or more properties related to the BCR repertoire of the individual patient; and
   (b) a diagnosis of the hypogammaglobulinemia patient whether or not the individual patient needs IgG-RT, optionally, the diagnosis is based on information associated with serum IgG levels or infections susceptibility;
(4) numerically encoding the training examples in the training dataset, comprising numerically encoding the one or more properties related to the BCR repertoire of the individual patient, and numerically encoding the diagnosis of the hypogammaglobulinemia patient of whether or not the individual patient needs IgG-RT;
(5) for a diagnostic model comprising a neural network that has a plurality of layers, each layer having a plurality of parameters, the layers comprising an input layer for receiving the numerically encoded one or more properties of the BCR repertoire and an output layer indicating a likelihood of requirement of IgG-RT, for one or more iterations of the training process:
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function to update the parameters of the layers of the diagnostic model, and
   (d) stopping the backpropagation after the loss function satisfies a criterion; and
(6) storing the updated set of parameters for the layers of the diagnostic model on the computer readable storage medium.

In some embodiments, in step (3) (a), the one or more properties related to the BCR repertoire of the individual patient is selected from 1) to 7),
1) the number or abundance of individual IgG clones in the BCR repertoire;
2) a diversity index value of the IgM antibody clones calculated by measuring the number and abundance of individual IgM antibody clones in the BCR repertoire;
3) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgG antibody clones that are most frequent in the BCR repertoire;
4) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgM antibody clones that are most frequent in the BCR repertoire;
5) a variable region (V) gene usage frequency in the antibody clones, optionally wherein the V gene is selected from the group consisting of IGHV4-30-2 heavy chain V gene, IGHV4-30-4 heavy chain V gene, IGHV3-23 heavy chain V gene, IGHV4-34 heavy chain V gene, and IGHV4-31 heavy chain V gene;
6) an average percent germline identity measured by comparing the variable (V) or joining (J) region in the BCR repertoire against a corresponding germline sequence; and
7) a median somatic nucleotide mutation frequency in the V region in the BCR repertoire, optionally wherein the V region comprises FR1, CDR1, FR2, CDR2, and/or FR3 regions.

One aspect of the present disclosure provides a diagnostic product for selecting a hypogammaglobulinemia patient for treatment with immunoglobulin replacement therapy (IgG-RT), wherein the diagnostic product is stored on a non-transitory computer readable medium and is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) obtaining a training dataset including a plurality of training examples, wherein each training example comprises the sequence information of an individual patient and a diagnosis of the individual patient whether or not the individual patient needs IgG-RT, optionally, the diagnosis is based on information associated with serum IgG levels or infections susceptibility;
(3) numerically encoding the training examples in the training dataset, comprising numerically encoding the sequence information of the individual patient, and numerically encoding the diagnosis of the individual patient of whether or not the individual patient needs IgG-RT;
(4) for a diagnostic model comprising a neural network that has a plurality of layers, each layer having a plurality of parameters, the layers comprising an input layer for receiving the numerically encoded sequence information and an output layer indicating a likelihood of requirement of IgG-RT, for one or more iterations of the training process:
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function, to update the parameters of the layers of the diagnostic model, and
   (d) stopping the backpropagation after the loss function satisfies a criterion; and
(5) storing the updated set of parameters for the layers of the diagnostic model on the computer readable storage medium.

Another aspect of the present disclosure provides a method of selecting a hypogammaglobulinemia patient for immunoglobulin replacement therapy (IgG-RT), comprising
(1) obtaining sequence information of at least 10,000 transcripts from the patient's sample comprising B cells, wherein each of the transcripts encodes a heavy chain of IgG or IgM or a portion thereof;
(2) providing the sequence information or information related to B cell receptor (BCR) repertoire of the patient obtained by processing the sequence information to the diagnostic product disclosed herein and operating the diagnostic product; and
(3) obtaining, from the diagnostic product, information related to whether or not the patient needs IgG-RT.

In some embodiments, the step (2) of characterizing B cell receptor (BCR) repertoire comprises analyzing the BCR repertoire by one or more steps selected from (a)-(f):
(a) measuring the number or abundance of individual antibody clones in the BCR repertoire;
(b) calculating a diversity index value of the antibody clones by measuring the number and abundance of individual antibody clones in the BCR repertoire;
(c) selecting at least 10 but no more than 30 antibody clones that are most frequent in the BCR repertoire and calculating a total frequency of the 10 to 30 frequent antibody clones;
(d) determining a variable region gene (V region) usage frequency in the antibody clones, optionally wherein the V is selected from the group consisting of IGHV4-30-2 heavy chain V region gene, IGHV4-30-4 heavy chain V region gene, IGHV3-23 heavy chain V region gene, IGHV4-34 heavy chain V region gene, and IGHV4-31 heavy chain V region gene;
(e) measuring a percent germline identity by comparing the V or J region in the BCR repertoire against a corresponding germline sequence; and
(f) determining the somatic mutation frequency in different regions along the V region.

In some embodiments, the method further comprises the step of treating the patient with IgG-RT when the patient is selected for IgG-RT.

The present disclosure also provides a method of treating a hypogammaglobulinemia patient, comprising administering immunoglobulin replacement therapy (IgG-RT) to the patient, wherein the patient has been selected for IgG-RT using the method disclosed herein.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIGS. 1A-1F. IgG and IgM antibody repertoire sequencing. (FIG. 1A) Antibody titer for IgG (left panel), IgM (middle panel), and IgA (right panel), for the patients who did and did not need IgG-RT. (FIG. 1B) Number of IgG and IgM antibody clones for the patients who did and did not need IgG-RT. (FIG. 1C) IgG and IgM antibody diversity indices for the patients who did and did not need IgG-RT. (FIG. 1D) Cumulative frequency of the top 20 IgG clones (each patient is a different color). The y-axis shows cumulative frequency, measured as percent of the total repertoire, while the x-axis shows the top 20 clones, ordered from the most to the least abundant. The right and left panels indicate patients who did and did not need IgG-RT, respectively. (FIG. 1E) Cumulative frequency of the top 20 IgM clones (each patient is a different color). (FIG. 1F) Heavy chain CDR3 amino acid length distribution, for IgG (left panel) and IgM (right panel).

FIGS. 2A-2J. Correlations between antibody repertoire and immune features. (FIGS. 2A and 2B) All-by-all correlation matrix of various antibody features and immune cell frequencies for patients who did not (FIG. 2A) and did (FIG. 2B) need IgG-RT. The numbers indicate Pearson correlation coefficients. Blue and red shadings indicate positive and negative correlation, respectively, as indicated in the legend in FIG. 2B. Only significant (p≤0.05) correlations are shown. (FIG. 2C-FIG. 2J) Scatter plots showing several significant correlations from FIG. 2A and FIG. 2B, for patients who did (right panels) and did not (left panels) need IgG-RT. The blue lines are linear regression lines while the gray shadings show the 95% confidence intervals around the fitted lines. P-values are indicated in black (p>0.05) or red (p≤0.05).

FIGS. 3A-3G. Antibody heavy chain V and J gene diversity. (FIG. 3A) Heatmaps showing the abundance of antibody clones with specific heavy chain V genes (y-axis) for the patients (x-axis) who did (right panel) and did not need IgG-RT (left panel), for IgG. The color indicates clone frequency per patient, as indicated by the legend. (FIG. 3B) Heat maps showing heavy chain V gene usage for IgM. (FIG. 3C) IgG heavy chain V genes that are present at different frequencies between the patients who did and did not need IgG-RT. Y-axis represents percent antibody clone with a given V gene. P-values are adjusted using the Benjamini-Hochberg method for multiple testing correction. (FIG. 3D) IgM heavy chain V gene usage difference between patient who did and did not need IgG-RT. (FIG. 3E) Boxplots showing percent nucleotide identity of V and J genes to germline sequences for all IgG clones. (FIG. 3F) Boxplots showing percent amino acid identity of V and J genes to germline sequences for all IgM clones. (FIG. 3G) V gene nucleotide mutation frequency in different regions, for IgG (left panel) and IgM (right panel). FR=framework: CDR=complementarity determining region. ns (not significant): p>0.05, *: p≤0.05, **: p≤0.01, ***: p≤0.001, ****: p≤0.0001.

FIGS. 4A-4C. IgG and IgM antibody repertoire sequencing. The analyses for FIG. 1A-IC were repeated after removing two higher titer donors (IgG titer >4 g/L or IgM titer >1 g/L). (FIG. 4A) Antibody titer for IgG, IgM and IgA, for the patients who did and did not need IgG-RT. (FIG. 4B) Number of IgG and IgM antibody clones. (FIG. 4C) IgG and IgM antibody diversity indices.

FIG. 5. Scatter plots showing several significant correlations between various antibody features and immune cell frequencies, for patients who did (right panels) and did not (left panels) need IgG-RT. The blue lines are linear regression lines while the gray shadings show the 95% confidence intervals around the fitted lines. P-values are indicated in black (p>0.05) or red (p≤0.05).

FIGS. 6A-6D. (FIG. 6A) Heatmaps showing the abundance of antibody clones with specific heavy chain J genes (y-axis) for the patients (x-axis) who did (right panel) and did not need IgG-RT (left panel), for IgG. The color indicates clone frequency per patient, as indicated by the legend. (FIG. 6B) Heatmaps showing heavy chain J gene usage for IgM. (FIG. 6C) Principal component analysis (PCA) using IgG heavy chain V gene usage frequencies. The data points represent individual patients, colored based on their need for IgG-RT, as indicated in the legend. (FIG. 6D) PCA using IgM V gene usage frequencies.

FIGS. 7A-7E. Antibody heavy chain V and J gene divergence from germline. (FIG. 7A) Boxplots showing percent nucleotide identity of V and J genes to germline sequences, for IgG. Repeated analysis of FIG. 3E, after removing two higher titer donors. (FIG. 7B) Boxplots showing percent amino acid identity of V and J genes to germline sequences, for IgM. Repeated analysis of FIG. 3F, after removing two higher titer donors. (FIG. 7C) V gene nucleotide mutation frequency in different regions, for IgG (left panel) and IgM (right panel). Repeated analysis of FIG. 3G, after removing two higher titer donors. (FIG. 7D) V gene amino acid mutation frequency in different regions, for IgG (left panel) and IgM (right panel). (FIG. 7E) Repeated analysis of 7D, with two higher titer donors removed. FR=framework: CDR=complementarity determining region. ns (not significant): p>0.05, *: p≤0.05, **: p≤0.01, ***: p≤0.001, ****: p≤0.0001.

FIG. 8. V gene nucleotide mutation frequency along the top five most common heavy chain V genes, for patients who did (right panels) and did not need IgG-RT (left panels), for IgG (top panels) and IgM (bottom panels). The x-axis indicates nucleotide position along the V gene, while the y-axis indicates nucleotide mutation frequency (number of nucleotide mutations per sequencing read). Individual donors are color coded, as indicated in the legend. The background shading indicates the framework (FR) and complementarity determining regions (CDR). The first 21 nucleotides (primer binding sites) were excluded from the analysis.

FIG. 9. V gene amino acid mutation frequency along the top five most common heavy chain V genes, for patients who did (right panels) and did not need IgG-RT (left panels), for IgG (top panels) and IgM (bottom panels). The x-axis indicates amino acid position along the V gene, while the y-axis indicates amino acid mutation frequency (number of amino acid mutations per sequencing read). Individual donors are color coded, as indicated in the legend. The background shading indicates the framework (FR) and complementarity determining regions (CDR). The first 7 amino acids (primer binding sites) were excluded from the analysis.

FIG. 10A-10B. IgG IGHV4-34 mutations. (FIG. 10A) Amino acid mutation frequency along IgG IGHV4-34, for patients who did (bottom panel) and did not need IgG-RT (top panel). The x-axis indicates amino acid position along the V gene, while the y-axis indicates amino acid mutation frequency (number of amino acid mutations per sequencing read). Individual donors are color coded, as indicated in the legend. The background shading indicates the framework (FR) and complementarity determining regions (CDR). The first 7 amino acids (primer binding sites) were excluded from the analysis. The protein sequence is shown, with the hydrophobic patch (AVY residues) indicated in red. (FIG. 10B) Mean mutation frequency at the IGHV4-34 hydrophobic patch (AVY residues). FIG. 10A discloses SEQ ID NOS 18 and 18, respectively, in order of appearance.

### 6. DETAILED DESCRIPTION OF THE INVENTION

### 6.2 Method of Selecting a Hypogammaglobulinemia Patient for Immunoglobulin Replacement Therapy (Ig-RT)

The present disclosure relates to a method of selecting a hypogammaglobulinemia patient for immunoglobulin replacement therapy (IgG-RT). In some embodiment, the method is used before immunoglobulin replacement therapy (IgG-RT). The method can use sequence information related to the patient's B cells. Accordingly, the method can further comprise the step of obtaining the sequence information. In some embodiments, the method comprises the step of sequencing the at least 10,000 transcripts of the patient, thereby providing the sequence information. In some embodiments, the sequence information is for at least 10,000 transcripts from the patient's sample comprising B cells. In some embodiments, the patient's sample comprises peripheral blood mononuclear cells (PBMCs).

In some embodiments, the method comprises:
(2) obtaining sequence information of at least 10,000 transcripts from the patient's sample comprising B cells, wherein each of the transcripts encodes a heavy chain of IgG or IgM or a portion thereof; and
(3) characterizing B cell receptor (BCR) repertoire of the patient by identifying antibody clones using the sequence information;
(4) analyzing the BCR repertoire;
(5) selecting a patient for IgG-RT based on the analysis of the BCR repertoire;
(6) providing information related to whether or not the patient needs IgG-RT.

In some embodiments, the method comprises:
(1) obtaining sequence information of at least 10,000 transcripts from the patient's sample comprising B cells, wherein each of the transcripts encodes a heavy chain of IgG or IgM or a portion thereof; and
(2) characterizing B cell receptor (BCR) repertoire of the patient by identifying antibody clones using the sequence information;
(3) analyzing the BCR repertoire by one or more selected from (a)-(f):
   (a) measuring the number or abundance of individual antibody clones in the BCR repertoire;
   (b) calculating a diversity index value of the antibody clones by measuring the number and abundance of individual antibody clones in the BCR repertoire;
   (c) selecting at least 10 but no more than 30 antibody clones that are most frequent in the BCR repertoire and calculating a total frequency of the 10 to 30 most frequent antibody clones;
   (d) determining a variable region gene (V region) usage frequency in the antibody clones, optionally wherein the V region gene is selected from the group consisting of IGHV4-30-2 heavy chain V region gene, IGHV4-30-4 heavy chain V region gene, IGHV3-23 gene, IGHV4-34 heavy chain V region gene, and IGHV4-31 heavy chain V region gene;
   (e) measuring a percent germline identity by comparing the V or J region in the BCR repertoire against a corresponding germline sequence; and
   (f) determining the somatic mutation frequency in different regions along the V region;
(4) selecting a patient for IgG-RT when one or more of (g)-(s) are satisfied:
   (g) the number of IgG clones in the BCR repertoire is greater than Tg, wherein Tg is at least 600;
   (h) the diversity index value of the IgM antibody clones is greater than Th, wherein Th is at least 250;
   (i) the total frequency of the most frequent 10 to 30 IgG antibody clones in the BCR repertoire is less than Ti, wherein Ti is at most 30%;
   (j) the total frequency of the most frequent 10 to 30 IgM antibody clones in the BCR repertoire is less than Tj, wherein Tj is at most 7%;
   (k) the frequency of IgG antibody clones with the IGHV4-30-2 heavy chain V region is less than Tk, wherein Tk is at most 0.3%;
   (l) the frequency of IgG antibody clones with the IGHV4-30-4 heavy chain V region is less than Tl, wherein Tl is at most 0.5%;
   (m) the frequency of IgG antibody clones with the IGHV3-23 heavy chain V region is greater than Tm, wherein Tm is at least 6%;
   (n) the frequency of IgG antibody clones with the IGHV4-34 heavy chain V region is greater than Tn, wherein Tn is at least 6%;
   (o) the frequency of IgM antibody clones with the IGHV4-31 heavy chain V region is less than To, wherein To is at most 0.2%;
   (p) IgG V gene average percent germline identity is greater than Tp, wherein Tp is at least 98%;
   (q) IgG J gene average percent germline identity is greater than Tq, wherein Tq is at least 98%;
   (r) the median somatic nucleotide mutation frequency in the FR1, CDR1, FR2, or CDR2 regions of IgG is lower than Tr, wherein Tr is at most 1; and
   (s) the median somatic nucleotide mutation frequency in the FR3 region of IgG is less than Ts, wherein Ts is at most 4;
(5) providing information related to whether or not the patient needs IgG-RT.
   6.5.1 Sequence Information from Patient's B Cell Transcripts

In some embodiments, the method disclosed herein uses sequence information of at least 10,000 transcripts from the patient's sample comprising B cells.

In some embodiments, the sequence information is obtained by sequencing a sample from the patient. In some embodiments, the sequence information is obtained from database.

In some embodiments, the sequence information is sequence information of at least 500 transcripts. In some embodiments, the sequence information is sequence information of at least 1000 transcripts. In some embodiments, the sequence information is sequence information of at least 5000 transcripts. In some embodiments, the sequence information is sequence information of at least 10,000 transcripts. In some embodiments, the sequence information is sequence information of at least 50,000 transcripts. In some embodiments, the sequence information is sequence information of at least 100,000, 500,000, 1000,000 or 10 million transcripts. In some embodiments, the sequence information is sequence information of more than 10 million transcripts.

In some embodiments, each of the transcripts encodes a heavy chain of IgG or IgM or a portion thereof. In some embodiments, each of the transcripts encodes a heavy chain of IgG or a portion thereof. In some embodiments, each of the transcripts encodes a heavy chain of IgM or a portion thereof. In some embodiments, transcripts as a group encode heavy chains of IgG and IgM. In some embodiments, each of the transcripts encodes a CDR3 of an IgG heavy chain. In some embodiments, each of the transcripts encodes a CDR3 of an IgM heavy chain. In some embodiments, each of the transcripts encodes a CDR3 of an IgG or IgM heavy chain.

### 6.5.2 Identification of Antibody Clones

In various embodiments, an antibody clone as used herein refers to homogeneous antibodies derived from a single B-cell which detects a single epitope within an immunogen. In some embodiments, clones are defined conservatively, where an antibody clone can include antibodies having one amino acid or 1-2 amino acid difference in the CDR3 region. In some embodiments, unique sequences are combined as a clone if they have 1 amino acid difference for CDR3H. In some embodiments, unique sequences are combined if they have 1-2 amino acids difference for CDR3H. In some embodiments, unique sequences are combined if they have 1 amino acid difference for 5-6 amino acid long CDR3H, or if they have 1-2 amino acid differences for >6 amino acid long CDR3H.

Antibody clones can be identified by various methods known in the art, such as single cell technologies, some involving microfluidic technologies.

In some embodiments, antibody clones can be identified using sequence information. Specifically, antibody clones can be identified by analyzing sequence information of transcripts from the patient's sample comprising B cells. In some embodiments, sequence information related to a heavy chain of IgG or IgM or a portion thereof is analyzed. In some embodiments, sequences corresponding to a CDR3 of an IgG or IgM heavy chain are used for identification of antibody clones. In some embodiments, sequences corresponding to a variable region of a heavy chain or a light chain of an IgG or IgM are used for identification of antibody clones.

### 6.5.3 Analysis of B Cell Repertoire

The method involves analysis of B cell repertoire in the patient.

In some embodiments, the number or abundance of individual antibody clones in the BCR repertoire is measured. In some embodiments, the number or abundance of at least 5 individual antibody clones in the BCR repertoire is measured. In some embodiments, the number or abundance of at least 10 individual antibody clones in the BCR repertoire is measured. In some embodiments, the number or abundance of at least 20 individual antibody clones in the BCR repertoire is measured. In some embodiments, the number or abundance of at least 50 individual antibody clones in the BCR repertoire is measured. In some embodiments, the number or abundance of at least 100 individual antibody clones in the BCR repertoire is measured. In some embodiments, the number or abundance of at least 500 individual antibody clones in the BCR repertoire is measured. In some embodiments, the number or abundance of at least 1000 individual antibody clones in the BCR repertoire is measured.

In some embodiments, a diversity index value of the antibody clones is calculated by measuring the number and abundance of individual antibody clones in the BCR repertoire. The diversity index value can be calculated by the method described in Example 6.1 (“Antibody diversity index”). Specifically, antibody diversity index can be calculated using the diversity function of the tcR package (version 2.3.2) in R version 4.1.2. The true diversity of an antibody repertoire X refers to the effective richness of that population: the number of equally common antibody clones that would be required to produce a repertoire with the same overall diversity as X. This value will increase with the number of antibody clones in the repertoire, as well as with the evenness with which these clones are distributed.

In some embodiments, the method comprises selecting at least 10 but no more than 30 antibody clones that are most frequent in the BCR repertoire and calculating a total frequency of the 10 to 30 most frequent antibody clones. In some embodiments, 10 most frequent antibody clones are selected. In some embodiments, 15 most frequent antibody clones are selected. In some embodiments, 20 most frequent antibody clones are selected. In some embodiments, 25 most frequent antibody clones are selected. In some embodiments, 30 most frequent antibody clones are selected. In some embodiments, 11, 12, 13, 14, 15, 16, 17, 18, 19, 20, 21, 22, 23, 24, 25, 26, 27, 28, 29 or 30 most frequent antibody clones are selected. In some embodiments, 10-15 most frequent antibody clones are selected. In some embodiments, 15-20 most frequent antibody clones are selected. In some embodiments, 20-25 most frequent antibody clones are selected. In some embodiments, 25-30 most frequent antibody clones are selected.

In some embodiments, the method comprises determining a variable region gene (V region) usage frequency in the antibody clones. In some embodiments, the method comprises determining usage frequency of the V region gene selected from the group consisting of IGHV4-30-2 heavy chain V region gene, IGHV4-30-4 heavy chain V region gene, IGHV3-23 gene, IGHV4-34 heavy chain V region gene, and IGHV4-31 heavy chain V region gene. In some embodiments, the method comprises determining usage frequency of the IGHV4-30-2 heavy chain V region gene. In some embodiments, the method comprises determining usage frequency of the IGHV4-30-4 heavy chain V region gene. In some embodiments, the method comprises determining usage frequency of the IGHV3-23 gene. In some embodiments, the method comprises determining usage frequency of the IGHV4-34 heavy chain V region gene. In some embodiments, the method comprises determining usage frequency of the IGHV4-31 heavy chain V region gene.

In some embodiments, the method comprises measuring a percent germline identity by comparing the V or J region in the BCR repertoire against a corresponding germline sequence. The germline V and J gene identity can be measured by mapping antibody nucleotide sequences to human V and J gene reference sequences. In some embodiments, the UBLAST alignment is used to assign V and J gene families and compute percent identity to germline sequences.

In some embodiments, the method comprises determining the somatic mutation frequency in different regions along the V region.

In some embodiments, the method comprises one or more selected from (a) to (f):
(a) measuring the number or abundance of individual antibody clones in the BCR repertoire;
(b) calculating a diversity index value of the antibody clones by measuring the number and abundance of individual antibody clones in the BCR repertoire;
(c) selecting at least 10 but no more than 30 antibody clones that are most frequent in the BCR repertoire and calculating a total frequency of the 10 to 30 most frequent antibody clones;
(d) determining a variable region gene (V region) usage frequency in the antibody clones, optionally wherein the V region gene is selected from the group consisting of IGHV4-30-2 heavy chain V region gene, IGHV4-30-4 heavy chain V region gene, IGHV3-23 gene, IGHV4-34 heavy chain V region gene, and IGHV4-31 heavy chain V region gene;
(e) measuring a percent germline identity by comparing the V or J region in the BCR repertoire against a corresponding germline sequence; and
(f) determining the somatic mutation frequency in different regions along the V region;

In some embodiments, one out of (a) to (f) is performed for analysis of BCR repertoire. In some embodiments, two out of (a) to (f) are performed for analysis of BCR repertoire. In some embodiments, three out of (a) to (f) are performed for analysis of BCR repertoire. In some embodiments, four out of (a) to (f) are performed for analysis of BCR repertoire. In some embodiments, five out of (a) to (f) are performed for analysis of BCR repertoire. In some embodiments, six out of (a) to (f) are performed for analysis of BCR repertoire.

In some embodiments, at least one out of (a) to (f) is performed for analysis of BCR repertoire. In some embodiments, at least two out of (a) to (f) are performed for analysis of BCR repertoire. In some embodiments, at least three out of (a) to (f) are performed for analysis of BCR repertoire. In some embodiments, at least four out of (a) to (f) are performed for analysis of BCR repertoire. In some embodiments, at least five out of (a) to (f) are performed for analysis of BCR repertoire. In some embodiments, all six of (a) to (f) are performed for analysis of BCR repertoire.

### 6.5.4 Selection of a Patient for IgG-RT

In various embodiments, the method comprises selecting a patient for IgG-RT based on the analysis of B cell repertoire. In some embodiments, a patient is selected for IgG-RT when the patient has a B cell repertoire similar to one or more patient who have been clinically demonstrated to require IgG-RT.

In some embodiments, the patient is selected for IgG-RT when the number of IgG clones in the BCR repertoire is greater than Threshold g (Tg), wherein Tg is at least 600. In some embodiments, Tg is 600, 650, 700, 750, 800, 850, 900, 950, 1,000, 1,050, or 1,100. In some embodiments, the patient is selected for IgG-RT when the number of IgG clones in the BCR repertoire is greater than Threshold g (Tg), wherein Tg is at least 600, 650, 700, 750, 800, 850, 900, 950, 1,000, 1,050, or 1,100. In some embodiments, the patient is selected for IgG-RT when the number of IgG clones in the BCR repertoire is greater than Threshold g (Tg), wherein Tg is a number between 600 and 2000, between 800 and 1500, between 1000 and 1500 or between 1100 and 1300. In some embodiments, Tg is a number between 700 and 1,200, between 700 and 1,100, between 700 and 1,000, between 800 and 1,100, between 800 and 1,000, between 900 and 1,100, between 900 and 1,000, between 800 and 900, or between 700 and 800.

In some embodiments, the patient is selected for IgG-RT when the diversity index value of the IgM antibody clones is greater than Threshold h (Th), wherein Th is at least 250. In some embodiments, Th is 250, 300, 350, 400, 450, 500, 550, or 600. In some embodiments, Th is a number between 250 and 650, between 300 and 650, between 350 and 650, between 400 and 650, between 450 and 650, between 250 and 600, between 300 and 600, between 350 and 600, between 400 and 600, between 450 and 600, between 250 and 550, between 300 and 550, between 350 and 550, between 400 and 550, between 450 and 550, between 250 and 500, between 300 and 500, between 350 and 500, between 400 and 500, or between 450 and 500.

In some embodiments, the patient is selected for IgG-RT the total frequency of the most frequent 10 to 30 IgG antibody clones in the BCR repertoire is less than Threshold i (Ti), wherein Ti is at most 30%. In some embodiments, Ti is 30%, 25%, or 20%. In some embodiments, Ti is a number between 20% and 35%, between 20% and 30%, between 20% and 25% or between 25% and 35%, or between 25% and 30%.

In some embodiments, the patient is selected for IgG-RT when the total frequency of the most frequent 10 to 30 IgM antibody clones in the BCR repertoire is less than Threshold j (Tj), wherein Tj is at most 7%. In some embodiments, Tj is 7%, 6.5%, 6%, 5.5%, 5%, 4.5%, or 4%. In some embodiments, Tj is a number between 4% and 7%, between 4% and 6%, between 4% and 5%, between 5% and 7%, between 5% and 6%, or between 6% and 7%.

In some embodiments, the patient is selected for IgG-RT when the frequency of IgG antibody clones with the IGHV4-30-2 heavy chain V region is less than Threshold k (Tk), wherein Tk is at most 0.3%. In some embodiments, Tk is 0.3%, 0.25%, 0.2%, or 0.15%. In some embodiments, Tk is between 0.15% and 0.3%, between 0.2% and 0.3%, between 0.25% and 0.3%, between 0.15% and 0.25%, between 0.15% and 0.2%, between 0.2% and 0.3%, or between 0.25% and 0.3%.

In some embodiments, the patient is selected for IgG-RT when the frequency of IgG antibody clones with the IGHV4-30-4 heavy chain V region is less than Threshold l (Tl), wherein Tl is at most 0.5%. In some embodiments, Tl is 0.5%, 0.45%, 0.4%, 0.35%, 0.3%, or 0.25%. In some embodiments, Tl is a number between 0.25% and 0.5%, between 0.25% and 0.45%, between 0.25% and 0.4%, between 0.25% and 0.35%, between 0.25% and 0.3%, between 0.3% and 0.5%, between 0.3% and 0.45%, between 0.3% and 0.4%, between 0.3% and 0.35%, between 0.35% and 0.45%, between 0.35% and 0.4%, between 0.4% and 0.5%, between 0.4% and 0.45%, or between 0.45% and 0.5%.

In some embodiments, the patient is selected for IgG-RT when the frequency of IgG antibody clones with the IGHV3-23 heavy chain V region is greater than Threshold m (Tm), wherein Tm is at least 6%. In some embodiments, Tm is 6%, 7%, 8%, 9%, or 10%. In some embodiments, Tm is a number between 6% and 10%, between 6% and 9%, between 6% and 8%, between 6% and 7%, between 7% and 10%, between 7% and 8%, between 8% and 10%, between 8% and 9%, or between 9% and 10%.

In some embodiments, the patient is selected for IgG-RT when the frequency of IgG antibody clones with the IGHV4-34 heavy chain V region is greater than Threshold n (Tn), wherein Tn is at least 6%. In some embodiments, Tn is 6%, 7%, 8%, 9%, or 10%. In some embodiments, Tn is a number between 6% and 10%, between 6% and 9%, between 6% and 8%, between 6% and 7%, between 7% and 10%, between 7% and 8%, between 8% and 10%, between 8% and 9%, or between 9% and 10%.

In some embodiments, the patient is selected for IgG-RT when the frequency of IgM antibody clones with the IGHV4-31 heavy chain V region is less than Threshold o (To), wherein To is at most 0.2%. In some embodiments, To is 0.2%, 0.15%, or 0.1%. In some embodiments, To is a number between 0.1% and 0.2%, between 0.1% and 0.15%, or between 0.15% and 0.2%.

In some embodiments, the patient is selected for IgG-RT when IgG V gene average percent germline identity is greater than Threshold p (Tp), wherein Tp is at least 98%. In some embodiments, Tp is 98%, 98.5% or 99%. In some embodiments, Tp is a number between 98% and 99%, between 98% and 98.5%, or between 98.5% and 99%.

In some embodiments, the patient is selected for IgG-RT when IgG J gene average percent germline identity is greater than Threshold q (Tq), wherein Tq is at least 98%. In some embodiments, Tq is 98%, 98.5% or 99%. In some embodiments, Tq is between 98% and 99%, between 98% and 98.5%, or between 98.5% and 99%.

In some embodiments, the patient is selected for IgG-RT when the median somatic nucleotide mutation frequency in the FR1, CDR1, FR2, or CDR2 regions of IgG is lower than Threshold (Tr), wherein Tr is at most 1. In some embodiments, Tr is 1, 0.8, or 0.6. In some embodiments, Tr is a number between 0.6 and 1, between 0.6 and 0.8, or between 0.8 and 1.

In some embodiments, the patient is selected for IgG-RT when the median somatic nucleotide mutation frequency in the FR3 region of IgG is less than Ts, wherein Ts is at most 4. In some embodiments, Ts is 4, 3.5, 3, 2.5, or 2. In some embodiments, Ts is a number between 2 and 4, between 2 and 3.5, between 2 and 3, between 2 and 2.5, between 2.5 and 4, between 2.5 and 3.5, between 2.5 and 3, between 3 and 4, between 3 and 3.5, or between 3.5 and 4.

In some embodiments, the patient is selected for IgG-RT when one or more of (g)-(s) are satisfied:
(g) the number of IgG clones in the BCR repertoire is greater than Tg, wherein Tg is at least 600;
(h) the diversity index value of the IgM antibody clones is greater than Th, wherein Th is at least 250;
(i) the total frequency of the most frequent 10 to 30 IgG antibody clones in the BCR repertoire is less than Ti, wherein Ti is at most 30%;
(j) the total frequency of the most frequent 10 to 30 IgM antibody clones in the BCR repertoire is less than Tj, wherein Tj is at most 7%;
(k) the frequency of IgG antibody clones with the IGHV4-30-2 heavy chain V region is less than Tk, wherein Tk is at most 0.3%;
(l) the frequency of IgG antibody clones with the IGHV4-30-4 heavy chain V region is less than Tl, wherein Tl is at most 0.5%;
(m) the frequency of IgG antibody clones with the IGHV3-23 heavy chain V region is greater than Tm, wherein Tm is at least 6%;
(n) the frequency of IgG antibody clones with the IGHV4-34 heavy chain V region is greater than Tn, wherein Tn is at least 6%;
(o) the frequency of IgM antibody clones with the IGHV4-31 heavy chain V region is less than To, wherein To is at most 0.2%;
(p) IgG V gene average percent germline identity is greater than Tp, wherein Tp is at least 98%;
(q) IgG J gene average percent germline identity is greater than Tq, wherein Tq is at least 98%;
(r) the median somatic nucleotide mutation frequency in the FR1, CDR1, FR2, or CDR2 regions of IgG is lower than Tr, wherein Tr is at most 1; and
(s) the median somatic nucleotide mutation frequency in the FR3 region of IgG is less than Ts, wherein Ts is at most 4.

In some embodiments, the patient is selected for IgG-RT when one criterion selected from (g) to (s) is satisfied. In some embodiments, the patient is selected for IgG-RT when two criteria selected from (g) to (s) are satisfied. In some embodiments, the patient is selected for IgG-RT when three criteria selected from (g) to (s) are satisfied. In some embodiments, the patient is selected for IgG-RT when four criteria selected from (g) to (s) are satisfied. In some embodiments, the patient is selected for IgG-RT when five criteria selected from (g) to (s) are satisfied. In some embodiments, the patient is selected for IgG-RT when six criteria selected from (g) to (s) are satisfied. In some embodiments, the patient is selected for IgG-RT when seven criteria selected from (g) to (s) are satisfied. In some embodiments, the patient is selected for IgG-RT when eight criteria selected from (g) to (s) are satisfied. In some embodiments, the patient is selected for IgG-RT when nine criteria selected from (g) to (s) are satisfied. In some embodiments, the patient is selected for IgG-RT when ten criteria selected from (g) to (s) are satisfied. In some embodiments, the patient is selected for IgG-RT when eleven criteria selected from (g) to (s) are satisfied. In some embodiments, the patient is selected for IgG-RT when twelve criteria selected from (g) to (s) are satisfied. In some embodiments, the patient is selected for IgG-RT when thirteen criteria selected from (g) to (s) are satisfied.

In some embodiments, the patient is selected for IgG-RT when at least two, at least three, at least four, at least five, at least six, at least seven, at least eight, at least nine, at least ten, at least eleven criteria, or at least twelve criteria selected from (g) to (s) are satisfied.

In some embodiments, the method is used a patient having been selected for having less than 5 g/L of serum IgG and more than 40/μL of peripheral B cells. In some embodiments, the patient has been selected for having less than 4.5 g/L of serum IgG and more than 35/μL of peripheral B cells. In some embodiments, the patient has been selected for having less than 4 g/L of serum IgG and more than 30/μL of peripheral B cells.

### 6.2 Providing Information Related to Whether or not the Patient Needs IgG-RT

The method disclosed herein involves providing information related to whether or not the patient needs IgG-RT. In some embodiments, the information is provided to the patient or a guardian of the patient. In some embodiments, the information is provided to a medical professional. In some embodiments, the information is provided as a report. In some embodiments, the information is provided in an online form, e.g., on the website or by email. In some embodiments, the information is provided on the screen.

In some embodiments, the person who received the information decides whether to treat the patient with IgG-RT or whether to receive IgG-RT. Thus, in some embodiments, the method further comprises treating the patient with IgG-RT. In some embodiments, the method comprises treating the patient with a therapy other than IgG-RT.

### 6.2 Method of Treatment

Another aspect of the present disclosure relates to treating a patient with hypogammaglobulinemia. In some embodiments, the treatment method comprises deciding whether to treat the patient with IgG-RT. In some embodiments, the patient has never been treated with IgG-RT.

In some embodiments, the method comprises the step of analyzing B cell repertoire of the patient as described here. In some embodiments, the method comprises treatment with IgG-RT only when the patient has been selected for IgG-RT using the method described herein. In some embodiments, the method comprises additional treatments known to be effective for treating hypogammaglobulinemia. In some embodiments, the method comprises treatment other than IgG-RT when the patient has not been selected for IgG-RT.

### 6.2 Diagnosis Product and Method of Diagnosis

In yet another aspect, the present disclosure provides a diagnostic product for selecting a hypogammaglobulinemia patient for treatment with immunoglobulin replacement therapy (IgG-RT). In some embodiments, the diagnostic product is designed to use the method for selecting a hypogammaglobulinemia patient for treatment with IgG-RT as described herein. In some embodiments, the diagnostic product is stored on a non-transitory computer readable medium. In some embodiments, the diagnostic product is a set of trained parameters of a machine-learning (ML) or artificial intelligence (AI) model.

In some embodiments the diagnostic product is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) characterizing B cell receptor (BCR) repertoire of each of the patients by identifying antibody clones based on the sequence information;
(3) obtaining a training dataset including a plurality of training examples, wherein each training example corresponds to a BCR repertoire of an individual patient and comprises:
   (a) one or more properties related to the BCR repertoire of the individual patient; and
   (b) a diagnosis of the hypogammaglobulinemia patient whether or not the individual patient needs IgG-RT, optionally, the diagnosis is based on information associated with serum IgG levels or infections susceptibility;
(4) numerically encoding the training examples in the training dataset, comprising numerically encoding the one or more properties related to the BCR repertoire of the individual patient, and numerically encoding the diagnosis of the hypogammaglobulinemia patient of whether or not the individual patient needs IgG-RT;
(5) for a diagnostic model comprising a neural network that has a plurality of layers, each layer having a plurality of parameters, the layers comprising an input layer for receiving the numerically encoded one or more properties of the BCR repertoire and an output layer indicating a likelihood of requirement of IgG-RT, for one or more iterations of the training process:
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function to update the parameters of the layers of the diagnostic model, and
   (d) stopping the backpropagation after the loss function satisfies a criterion; and
(6) storing the updated set of parameters for the layers of the diagnostic model on the computer readable storage medium.

In some embodiments, in step (3) (a), the one or more properties related to the BCR repertoire of the individual patient is selected from 1) to 7),
1) the number or abundance of individual IgG clones in the BCR repertoire;
2) a diversity index value of the IgM antibody clones calculated by measuring the number and abundance of individual IgM antibody clones in the BCR repertoire;
3) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgG antibody clones that are most frequent in the BCR repertoire;
4) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgM antibody clones that are most frequent in the BCR repertoire;
5) a variable region (V) gene usage frequency in the antibody clones, optionally wherein the V gene is selected from the group consisting of IGHV4-30-2 heavy chain V gene, IGHV4-30-4 heavy chain V gene, IGHV3-23 heavy chain V gene, IGHV4-34 heavy chain V gene, and IGHV4-31 heavy chain V gene;
6) an average percent germline identity measured by comparing the variable (V) or joining (J) region in the BCR repertoire against a corresponding germline sequence; and
7) a median somatic nucleotide mutation frequency in the V region in the BCR repertoire, optionally wherein the V region comprises FR1, CDR1, FR2, CDR2, and/or FR3 regions.

In some embodiments, each of the one or more properties related to the BCR repertoire of the individual patient selected from 1) to 7) is numerically encoded as a scalar, a vector, or a tensor. In some embodiments, one or any combination of the one or more properties related to the BCR repertoire of the individual patient is input to the neural network as one or more scalars concatenated into a vector or tensor.

In some embodiments, the diagnosis of the hypogammaglobulinemia patient of whether or not the individual patient needs IgG-RT is numerically encoded as a categorical variable. In some embodiments, the categorical variable is a binary variable that is encoded as a non-zero value (e.g., value of “1”) if IgG-RT was required for the individual patient and encoded as a zero value if IgG-RT was not required for the individual patient.

In some embodiments, the diagnosis of the hypogammaglobulinemia patient of whether or not the individual patient needs IgG-RT is numerically encoded as a continuous variable. The continuous variable indicates a degree to which IgG-RT was required for the individual patient.

In some embodiments, the loss function for a current iteration of the training process is one or more of an L1 norm, a L2 norm, a L-infinity norm, a cross-entropy loss. In some embodiments, the cross-entropy loss is given by:

$${L}_{t} = {\sum }_{i = 1}^{{n}_{t}} {y}_{i , 0} \cdot log {\hat{y}}_{i , 0} + {y}_{i , 1} \cdot log {\hat{y}}_{i , 1}$$

where y_{i,0} is 1 if the diagnosis for individual patient of training example i for the current iteration indicates that no IgG-RT is required and 0 otherwise, y_{i,1} if 1 if the diagnosis for the individual patient indicates that IgG-RT is required, ŷ_{i,1} is the estimated likelihood generated by the neural network that the individual patient requires IgG-RT, and ŷ_{i,0} is the estimated likelihood that the individual patient does not require IgG-RT (e.g., 1−ŷ_{i,1}). However, it is appreciated that any other appropriate loss function can be used.

In some embodiments, the backpropagation for a current iteration is performed by computing a gradient of the computed loss for the iteration with respect to the parameter space of the neural network and updating the previous set of parameters by a factor of the computed gradient.

In some embodiments, the architecture of the neural network is configured as an artificial neural network (ANN), a feed-forward neural network, a deep neural network (DNN), a recurrent neural network (RNN), a transformer neural network with one or more attention layers, and the like. In some embodiments, the number of parameters of the neural network is greater than 1,000 parameters, 10,000 parameters, 100,000 parameters, 1 million parameters, 1 billion parameters, 10 billion parameters, 100 billion parameters, 1 trillion parameters.

In another aspect, the present disclosure provides a diagnostic product for selecting a hypogammaglobulinemia patient for treatment with immunoglobulin replacement therapy (IgG-RT), wherein the diagnostic product is stored on a non-transitory computer readable medium and is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) obtaining a training dataset including a plurality of training examples, wherein each training example comprises the sequence information of an individual patient and a diagnosis of the individual patient whether or not the individual patient needs IgG-RT, optionally, the diagnosis is based on information associated with serum IgG levels or infections susceptibility;
(3) numerically encoding the training examples in the training dataset, comprising numerically encoding the sequence information of the individual patient, and numerically encoding the diagnosis of the individual patient of whether or not the individual patient needs IgG-RT;
(4) for a diagnostic model comprising a neural network that has a plurality of layers, each layer having a plurality of parameters, the layers comprising an input layer for receiving the numerically encoded sequence information and an output layer indicating a likelihood of requirement of IgG-RT, for one or more iterations of the training process;
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function, to update the parameters of the layers of the diagnostic model, and
   (d) stopping the backpropagation after the loss function satisfies a criterion; and
(5) storing the updated set of parameters for the layers of the diagnostic model on the computer readable storage medium.

In some embodiments, the sequence information of the individual patient is numerically encoded as a scalar, a vector, or a tensor.

In some embodiments, the diagnosis of the hypogammaglobulinemia patient of whether or not the individual patient needs IgG-RT is numerically encoded as a categorical variable. In some embodiments, the categorical variable is a binary variable that is encoded as a non-zero value (e.g., value of “1”) if IgG-RT was required for the individual patient and encoded as a zero value if IgG-RT was not required for the individual patient.

In some embodiments, the diagnosis of the hypogammaglobulinemia patient of whether or not the individual patient needs IgG-RT is numerically encoded as a continuous variable. The continuous variable indicates a degree to which IgG-RT was required for the individual patient.

In some embodiments, the loss function for a current iteration of the training process is one or more of an L1 norm, a L2 norm, a L-infinity norm, a cross-entropy loss. In some embodiments, the cross-entropy loss is given by:

$${L}_{t} = {\sum }_{i = 1}^{{n}_{t}} {y}_{i , 0} \cdot log {\hat{y}}_{i , 0} + {y}_{i , 1} \cdot log {\hat{y}}_{i , 1}$$

where y_{i,0} is 1 if the diagnosis for individual patient of training example i for the current iteration indicates that no IgG-RT is required and 0 otherwise, y_{i,1} if 1 if the diagnosis for the individual patient indicates that IgG-RT is required, ŷ_{i,1} is the estimated likelihood generated by the neural network that the individual patient requires IgG-RT, and ŷ_{i,0} is the estimated likelihood that the individual patient does not require IgG-RT (e.g., 1−ŷ_{i,1}). However, it is appreciated that any other appropriate loss function can be used.

In some embodiments, the backpropagation for a current iteration is performed by computing a gradient of the computed loss for the iteration with respect to the parameter space of the neural network and updating the previous set of parameters by a factor of the computed gradient.

In some embodiments, the architecture of the neural network is configured as an artificial neural network (ANN), a feed-forward neural network, a deep neural network (DNN), a recurrent neural network (RNN), a transformer neural network with one or more attention layers, and the like. In some embodiments, the number of parameters of the neural network is greater than 1,000 parameters, 10,000 parameters, 100,000 parameters, 1 million parameters, 1 billion parameters, 10 billion parameters, 100 billion parameters, 1 trillion parameters.

In one aspect, the present disclosure provides a method of using the diagnostic product for selecting a hypogammaglobulinemia patient for treatment with immunoglobulin replacement therapy (IgG-RT).

In some embodiments, the method comprises:
(1) obtaining sequence information of at least 10,000 transcripts from the patient's sample comprising B cells, wherein each of the transcripts encodes a heavy chain of IgG or IgM or a portion thereof;
(2) providing the sequence information or information related to B cell receptor (BCR) repertoire of the patient obtained by processing the sequence information to the diagnostic product disclosed herein and operating the diagnostic product; and
(3) obtaining, from the diagnostic product, information related to whether or not the patient needs IgG-RT.

In some embodiments, the trained parameters of the neural network stored in the diagnostic product is applied to the sequence information or information related to B cell receptor (BCR) repertoire to generate a likelihood of whether the subject needs IgG-RT.

In some embodiments, the step (2) of characterizing B cell receptor (BCR) repertoire comprises analyzing the BCR repertoire by one or more steps selected from (a)-(f):
(a) measuring the number or abundance of individual antibody clones in the BCR repertoire;
(b) calculating a diversity index value of the antibody clones by measuring the number and abundance of individual antibody clones in the BCR repertoire;
(c) selecting at least 10 but no more than 30 antibody clones that are most frequent in the BCR repertoire and calculating a total frequency of the 10 to 30 frequent antibody clones;
(d) determining a variable region gene (V region) usage frequency in the antibody clones, optionally wherein the V is selected from the group consisting of IGHV4-30-2 heavy chain V region gene, IGHV4-30-4 heavy chain V region gene, IGHV3-23 heavy chain V region gene, IGHV4-34 heavy chain V region gene, and IGHV4-31 heavy chain V region gene;
(e) measuring a percent germline identity by comparing the V or J region in the BCR repertoire against a corresponding germline sequence; and
(f) determining the somatic mutation frequency in different regions along the V region.

In some embodiments, the method further comprises the step of treating the patient with IgG-RT when the patient is selected for IgG-RT.

In yet another aspect, the present disclosure provides a method of diagnosing a hypogammaglobulinemia patient for treatment with immunoglobulin replacement therapy (IgG-RT). In some embodiments, the method comprises:
(1) obtaining sequence information of at least 10,000 transcripts from the patient's sample comprising B cells, wherein each of the transcripts encodes a heavy chain of IgG or IgM or a portion thereof;
(2) obtaining parameters of a trained neural network configured to receive the sequence information of a patient or the BCR repertoire of the patient and generate a likelihood of whether the patient needs IgG-RT;
(3) providing the sequence information or information related to B cell receptor (BCR) repertoire of the patient obtained by processing the sequence information to the trained neural network disclosed herein; and
(4) obtaining, from the trained neural network, information related to whether or not the patient needs IgG-RT.

In some embodiments the neural network is trained by a method comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) characterizing B cell receptor (BCR) repertoire of each of the patients by identifying antibody clones based on the sequence information;
(3) obtaining a training dataset including a plurality of training examples, wherein each training example corresponds to a BCR repertoire of an individual patient and comprises:
   (a) one or more properties related to the BCR repertoire of the individual patient; and
   (b) a diagnosis of the hypogammaglobulinemia patient whether or not the individual patient needs IgG-RT, optionally, the diagnosis is based on information associated with serum IgG levels or infections susceptibility;
(4) numerically encoding the training examples in the training dataset, comprising numerically encoding the one or more properties related to the BCR repertoire of the individual patient, and numerically encoding the diagnosis of the hypogammaglobulinemia patient whether or not the individual patient needs IgG-RT;
(5) for a diagnostic model comprising a neural network that has a plurality of layers, each layer having a plurality of parameters, the layers comprising an input layer for receiving the numerically encoded one or more properties of the BCR repertoire and an output layer indicating a likelihood of requirement of IgG-RT, for one or more iterations of the training process:
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function to update the parameters of the layers of the diagnostic model, and
   (d) stopping the backpropagation after the loss function satisfies a criterion.

In some embodiments the neural network is trained by a method comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) obtaining a training dataset including a plurality of training examples, wherein each training example comprises the sequence information of an individual patient and a diagnosis of the individual patient whether or not the individual patient needs IgG-RT, optionally, the diagnosis is based on information associated with serum IgG levels or infections susceptibility;
(3) numerically encoding the training examples in the training dataset, comprising numerically encoding the sequence information of the individual patient, and numerically encoding the diagnosis of the individual patient whether or not the individual patient needs IgG-RT;
(4) for a diagnostic model comprising a neural network that has a plurality of layers, each layer having a plurality of parameters, the layers comprising an input layer for receiving the numerically encoded sequence information and an output layer indicating a likelihood of requirement of IgG-RT, for one or more iterations of the training process:
   (a) for a set of training examples for the current iteration, applying parameters of the neural network to generate estimated likelihoods for the set of training examples,
   (b) computing a loss function indicating a difference between the estimated likelihoods and the numerically encoded diagnoses for the set of training examples for the current iteration,
   (c) repeatedly backpropagating one or more error terms obtained from the loss function, to update the parameters of the layers of the diagnostic model, and
   (d) stopping the backpropagation after the loss function satisfies a criterion.

By training and deploying a neural network and a diagnostic product storing the parameters of same can learn patterns and features of a BCR repertoire and/or sequence information that is indicative of the maturity or immaturity of the patient's immune cells and further more whether the patient would require IgG-RT to treat hypogammaglobulinemia.

### 7. EXAMPLES

### 6.2 Experimental Methods for Identifying Differences of Hypogammaglobulinemia Patients Who Did or Did not Need Immunoglobulin Replacement Therapy

### Sample Collection

Patients were identified from the adult outpatient immunodeficiency clinic of the University of Freiburg for having decreased levels of serum IgG (<4 g/L) and remaining peripheral B cells of >40/μl. In the case of patients with the need for IgG-RT (those who had recurrent infections of the respiratory tract, n=15), hypogammaglobulinemia was evaluated using retrospective data from the time of diagnosis (before starting regular IgG-RT). Hypogammaglobulinemia patients that did not have recurrent respiratory tract infections (n=10) were not prescribed IgG-RT. The patient's infection history, other non-infectious diagnoses, and their ability to respond to vaccines are provided in Table 1.

The participating individuals donated blood samples after signing an informed written consent. PBMCs from the donated blood samples were isolated using Ficoll/Pancoll density gradient centrifugation under sterile conditions, following standard protocols. The harvested PBMCs (9-17×10⁶ cells/ml) in freezing medium (heat-inactivated 90% fetal bovine serum (FBS)+10% dimethyl sulfoxide (DMSO)) were stored in liquid nitrogen until further processing.

### Flow Cytometry

Red blood cells from 500 μl whole blood were lysed for 10 minutes at 4° C. with ammonium chloride, washed twice with phosphate-buffered saline (PBS)+2% FBS, and stained with anti-CD19 (APC-Cy7, HIB19, Biolegend), anti-CD27 (BV421, M-T271, Biolegend), anti-IgD (PE, IA6-2, Biolegend), anti-IgA (FITC, goat IgG, Southern Biotech), and anti-IgG (AF700, G18-145, BD Biosciences) for 20 minutes at room temperature. Subsequent fixation (Optilyse B, Beckman Coulter) for 20 minutes at room temperature was followed by another washing step with PBS+2% FBS. Stained cells were measured with Navios Flow Cytometer (Beckman-Coulter) and analyzed with Kaluza Analysis Software (Beckman-Coulter).

### Antibody Repertoire Sequencing

The harvested PBMCs were thawed into media (RPMI+10% FBS) and counted on a Cellometer K2 (Nexcelom). The cells were pelleted by centrifugation and RNA was extracted using a NucleoSpin RNA Plus kit (Macherey-Nagel) according to manufacturer's instructions. To amplify heavy chain variable regions for deep sequencing, tailed-end RT-PCR was performed on the extracted RNA. At the 5′ end, a pool of variable region primers with Illumina adapters was used, and at the 3′ end, a constant region primer (for IgG or IgM) with a sample-specific index sequence and Illumina adapter was used (Table 2); IgG and IgM sequences were amplified in separate reactions. The PCR product was run on an agarose gel, extracted, purified, and quantified using a KAPA quantitative PCR Illumina Library Quantification Kit (1069, Roche). The libraries were sequenced as previously described on a MiSeq (Illumina) at a library concentration of 9 μM with a 255-cycle forward read and a 255-cycle reverse read (see Table 2 for sequencing primers). Sequencing data are available in the Short Read Archive under project identifier PRJNA876301.

### Antibody Sequence Analysis

The antibody repertoire libraries were sequenced to an average of 28,901 reads (range: 13,064-45,080 reads). Sequence analysis was performed. Briefly, the expected number of errors (E) for a read was calculated from its Phred scores and discarded reads with E>2. After error filtering, up to 15,000 reads were randomly sampled from each sample for further analysis. Applicant verified that our findings were consistent across multiple rounds of random read sampling (data not shown). IMGT immunoglobulin sequences were processed to generate position-specific sequences matrices (PSSMs) for each framework/CDR junction. These PSSMs were used to identify framework/CDR junctions for each of the nucleotide sequences. Python scripts were then used to translate the sequences. Reads were required to have a valid predicted CDR3 sequence. Applicant then defined antibody “clones” conservatively, where unique sequences were combined if they had 1 amino acid difference for 5-6 amino acid long CDR3H, or if they had 1-2 amino acid differences for >6 amino acid long CDR3H. Only clones with at least two sequencing reads were included in the analysis.

UBLAST was run using the nucleotide sequences as queries and V and J gene sequences from the IMGT database as the reference sequences. The UBLAST alignment with the lowest E-value was used to assign V and J gene families and compute percent identity to germline. The IgG sample for patient CVID-1712-01 had low sequence quality and was excluded from analysis.

### Antibody Diversity Index

Antibody diversity index was calculated using the diversity function of the tcR package (version 2.3.2) in R version 4.1.2. The true diversity of an antibody repertoire X refers to the effective richness of that population: the number of equally common antibody clones that would be required to produce a repertoire with the same overall diversity as X. This value will increase with the number of antibody clones in the repertoire, as well as with the evenness with which these clones are distributed.

### Correlation Analysis

The data used for the correlation analysis are in Table 1. Pearson correlation analysis was performed using the cor function of the corrplot package (version 0.92) using the “pairwise.complete.obs” option, in R version 4.1.2. Correlations with p≤0.05 were considered significant.

### Variable (V) Gene Usage and Mutation Frequency

To identify antibody V gene identity, sequencing fasta files were mapped to human V gene reference sequences (release 202243-1, 24 Oct. 2022) from IMGT, using USEARCH version v8.1.1916M_i86linux64 (options: -usearch_local-mismatch -1 -id 0.5 -evalue 1e-3). The IMGT antibody numbering system was used to identify CDR and framework regions along V genes (which was also used to determine CDR3H length). Principal component analysis (PCA) was performed using log 2-transformed V gene usage frequencies. Wilcoxon rank sum tests were used to compare V gene usage frequencies between donors who did and did not need IgG-RT. P-values were adjusted for the number of V genes tested using the Benjamini-Hochberg method. The number of mismatches along V genes were tallied using custom Perl scripts and visualized using ggplot2 in R. The first 21 nucleotides (7 amino acids) of V genes were the PCR primer binding sites for preparing the antibody sequencing libraries. Any mutations in this region could not be accurately measured and thus the region was excluded from the V gene mutation frequency analysis.

### 6.2 Differences of Hypogammaglobulinemia Patients Who Did or Did not Need Immunoglobulin Replacement Therapy

### Patient Cohorts

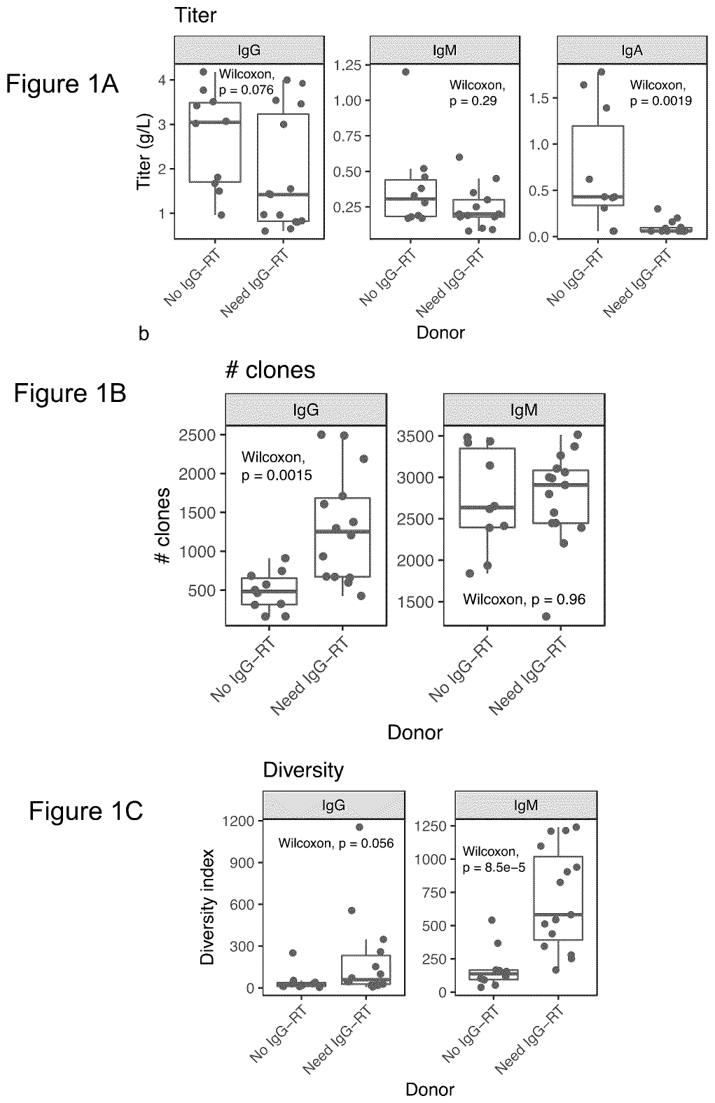

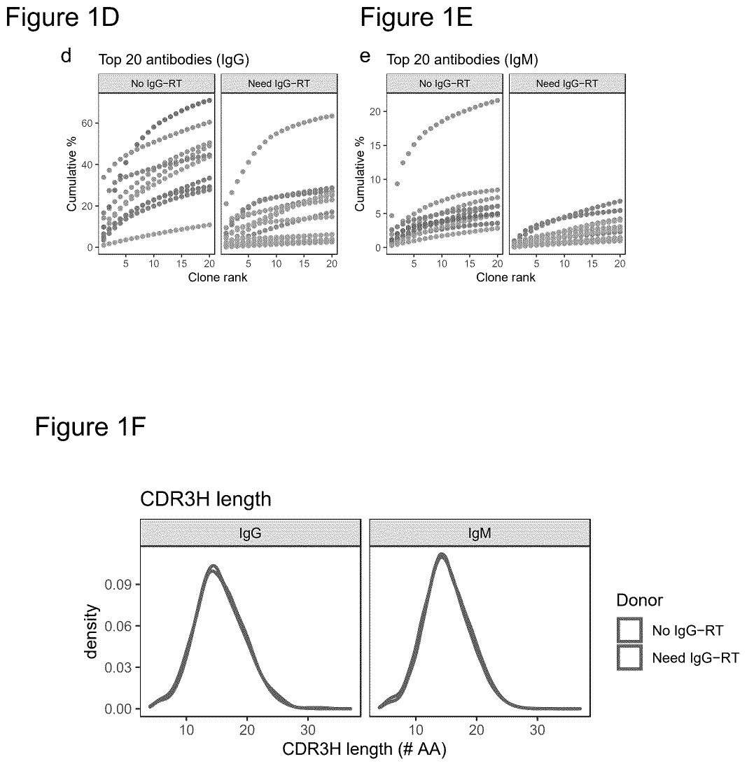

25 patients with low IgG serum concentrations were recruited and 15 of them needed IgG-RT (male: 3: female: 12), and 10 did not need IgG-RT (male: 8: female: 2), based on their susceptibility to infection (Table 1). On average, the patients who needed IgG-RT had 1.86 g/L IgG prior to IgG-RT (standard deviation, SD=1.31), 0.24 g/L IgM (SD=0.15), and 0.10 g/L IgA (SD=0.072) in serum, while the patients who did not need IgG-RT had 2.69 g/L IgG (SD=1.11), 0.39 g/L IgM (SD=0.31), and 0.71 g/L IgA (SD=0.64) in serum (FIG. 1A); the serum IgA titers were significantly different between the two groups (p=0.0019). The patients who needed IgG-RT had a comparable amount of CD19⁺ B cells (mean=221.3 cells/μl, SD=146.1) compared to the patients without the need of IgG-RT (208.9 cells/μl, SD=279.5: p=0.24). Vaccine responses against various pathogens (e.g., tetanus, diphtheria, and pneumococcal polysaccharide) were observed for most patients that did not need IgG-RT compared to patients who did need IgG-RT. Furthermore, autoimmune manifestations, chronic infections, and other complications were more common in patients who needed IgG-RT (Table 1).

### Antibody Repertoire Sequencing

IgG and IgM antibody repertoire sequencing of the heavy chain immunoglobulin for both patient cohorts from isolated PBMCs (IgA repertoires could not be investigated in this study due to low or absent IgA-memory B cell counts in most of the patients in need of IgG-RT; FIG. 1A, Table 1) was performed. Antibody “clones” were defined conservatively, where unique sequences were combined if they had one amino acid difference within 5-6 amino acid long CDR3H (complementarity-determining region 3 heavy chain), or if they had one to two amino acid differences for >6 amino acid long CDR3H. Only clones with at least two sequencing reads were included in the analysis. Interestingly, the patients who needed IgG-RT had a significantly higher number of IgG clones (mean=1,310 clones) than the patients who did not need IgG-RT (mean=483 clones: p=0.0015) (FIG. 1B). On the other hand, there was no significant difference in the number of IgM clones between those who did (mean=2,760 clones) and those who did not need IgG-RT (mean=2,733 clones: p=0.96).

To further examine the IgG and IgM antibody repertoires, the true diversity index was measured, which considers the abundance of individual antibody clones in addition to the number of clones. The true diversity of an antibody repertoire X refers to the effective richness of that population: the number of equally common antibody clones that would be required to produce a repertoire with the same overall diversity as X. This value will increase with the number of antibody clones in the repertoire, as well as with the evenness with which these clones are distributed. Relative to those who did not need IgG-RT, the patients who needed IgG-RT had significantly higher IgM diversity index (p=8.5×10⁻⁵) (FIG. 1C).

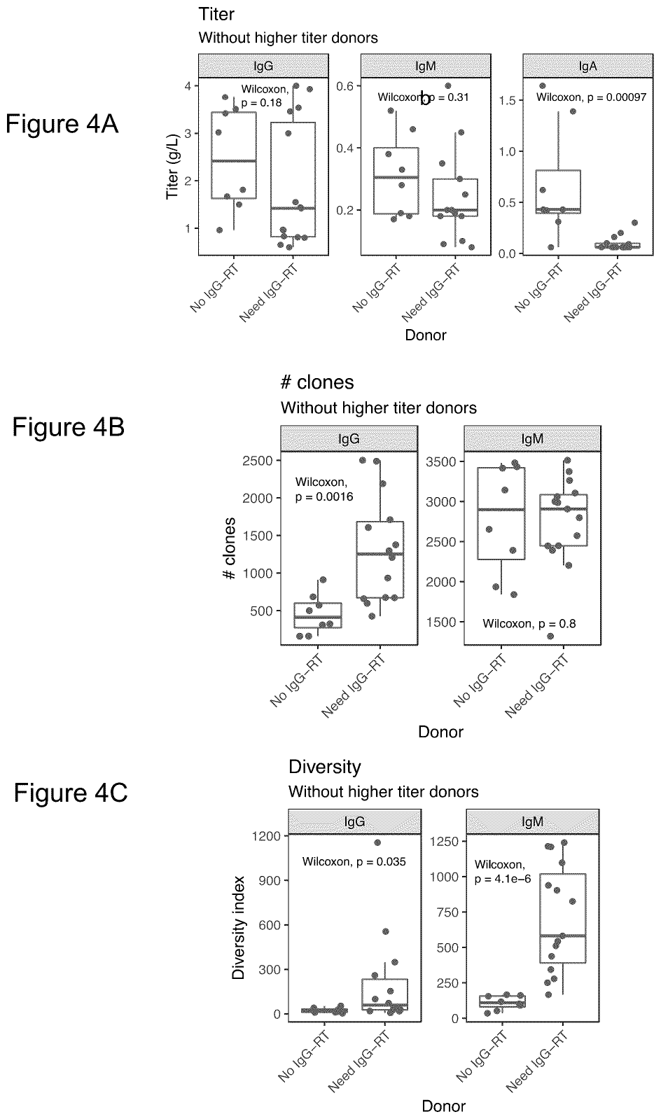

Among the donors who did not need IgG-RT, one donor had an IgG titer of 4.18 g/L and an additional donor had an IgM titer of 1.2 g/L (FIG. 1A, Table 1). To ensure that these donors with higher antibody titer were not driving the differences in antibody clone counts and diversity, we removed these donors from the datasets (FIG. 4A) and repeated the above analyses. We observed the same differences, where the donors who needed IgG-RT had significantly higher number of IgG clones (p=0.0016: FIG. 4B) and a higher IgM diversity index (p=4.1×10⁻⁶: FIG. 4C).

Visualizations of the frequencies of the top 20 antibody clones showed that patients who needed IgG-RT tended to have less oligoclonal IgG and IgM repertoires (FIGS. 1D, 1E). On average, the top 20 IgG clones made up 19.5% and 42.1% of the total repertoire for the patients who did and the ones who did not need IgG-RT, respectively, indicating lower IgG oligoclonality for the former cohort (p=0.0015). Similarly, the top 20 IgM clones accounted for on average of 2.65% and 7.06% of the repertoire for the patients who did and did not need IgG-RT, respectively, indicating lower IgM oligoclonality for the former cohort (p=0.0014).

Applicant further examined the distribution of the CDR3H amino acid sequence lengths, another feature that may provide insight into the composition of the antibody repertoire. However, both patient cohorts had normally distributed heavy chain CDR3 lengths with a median of 15 amino acids, for both IgG and IgM (FIG. 1F).

Together, these data show that the patients who needed IgG-RT had more IgG clones, a higher IgM diversity index, and lower IgG and IgM oligoclonality, consistent with more diverse antibody repertoires.

### Correlations Between Antibody Repertoire and Immune Features

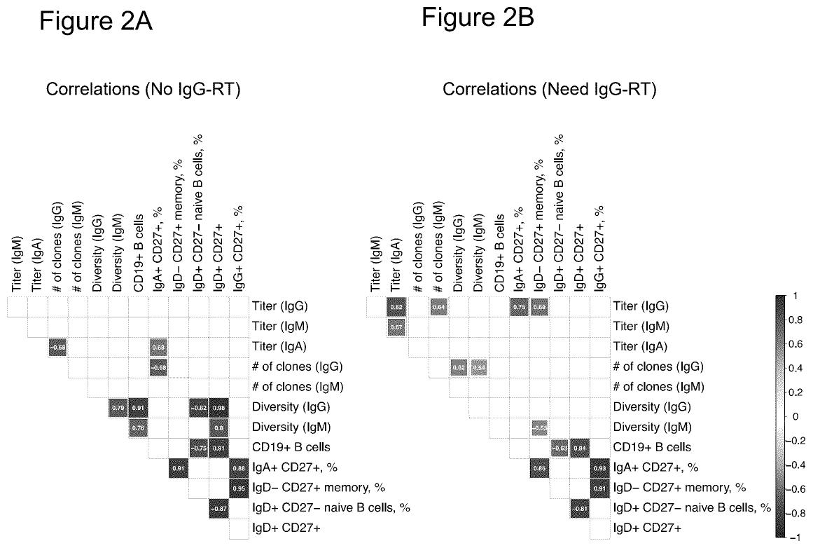

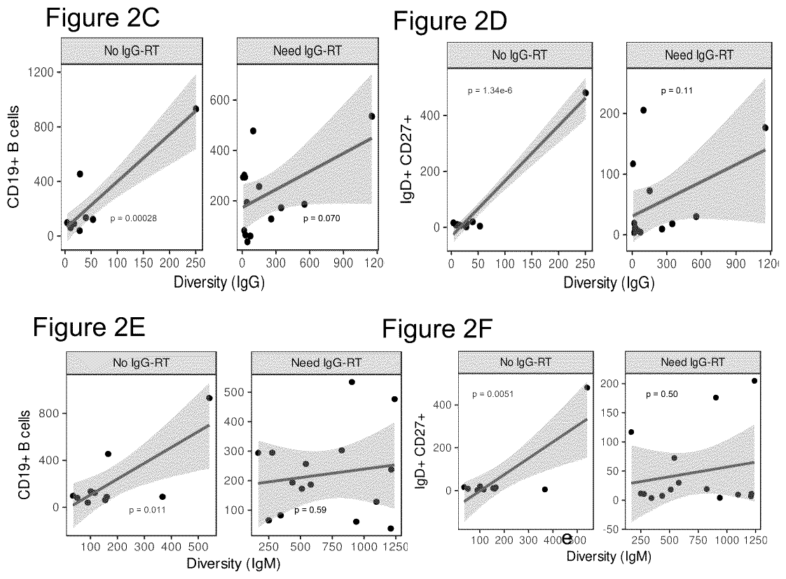

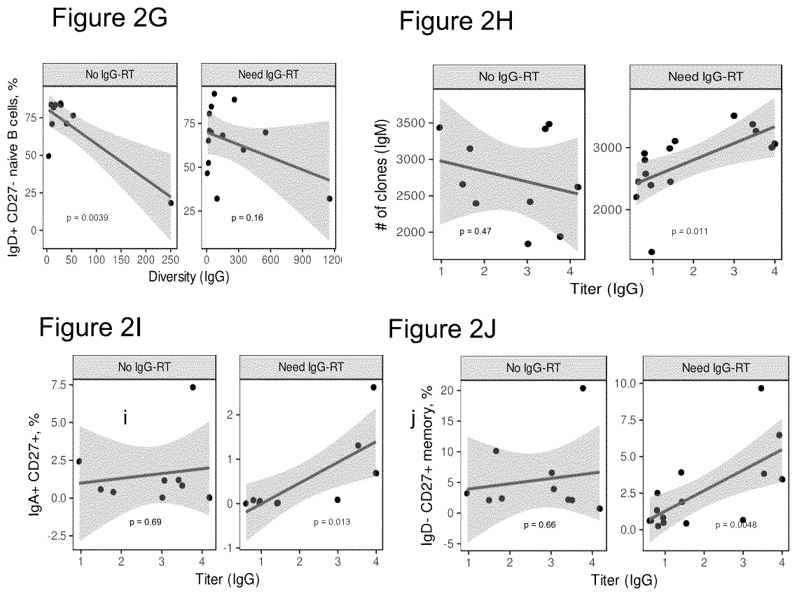

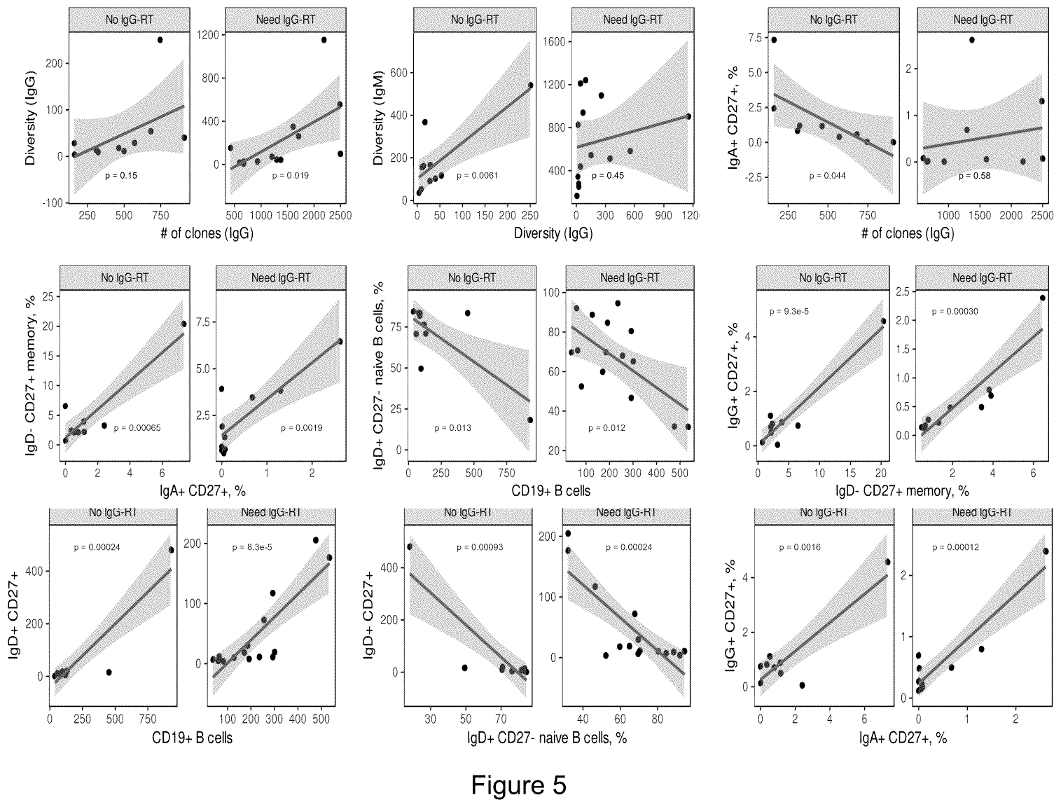

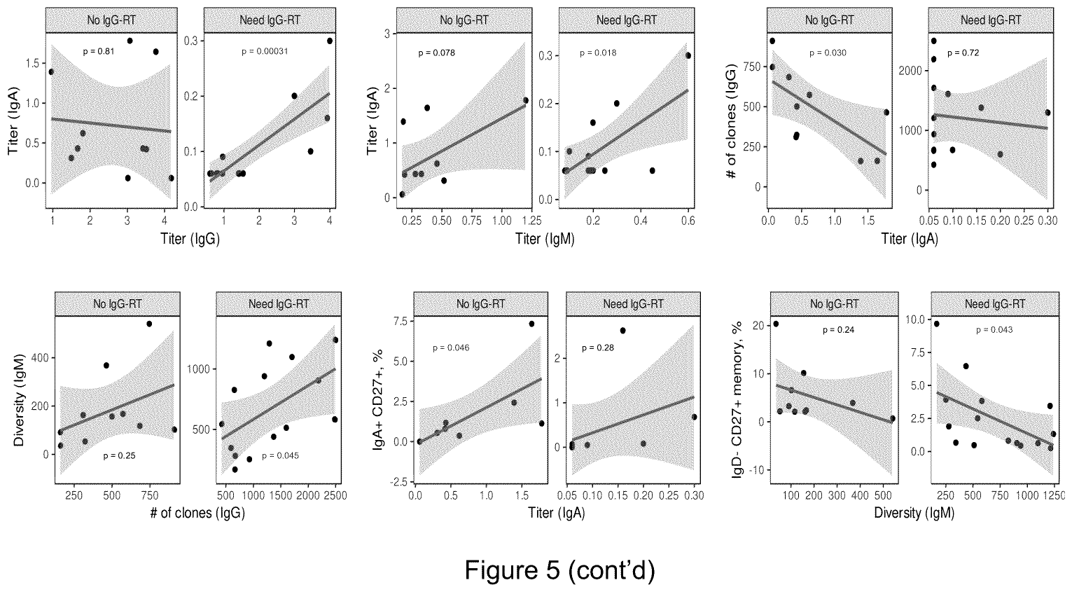

Next, the interplay between different features of the antibody repertoires and various immune parameters was studied. For both patient cohorts, the frequency of different B cell subtypes was measured by flow cytometry. Then, experiments were performed for an all-by-all correlation analysis of antibody titer, clone count, diversity, and abundance of different B cell subtypes for the patients who did and did not need IgG-RT (FIGS. 2A-2J, 5: Table 1).

For patients who did not need IgG-RT, IgG diversity positively correlated with the frequency of CD19+ B cells (Pearson correlation coefficient, r=0.91, p=0.00028) and IgD+CD27+ B cells (r=0.98, p=1.34×10⁻⁶) (FIGS. 2A, 2C, 2D). In the same patient cohort, IgM diversity also positively correlated with the frequency of CD19+ B cells (r=0.76, p=0.011) and IgD+CD27+ B cells (r=0.8, p=0.0051) (FIGS. 2A, 2E, 2F). IgG diversity negatively correlated with the frequency of IgD+CD27− naïve B cells (r=−0.82, p=0.0039) (FIGS. 2A, 2G). These correlations of IgG and IgM diversity with B cell frequencies were not observed in the patients who needed IgG-RT (FIG. 2B).

For patients who needed IgG-RT, the IgG titer correlated with the number of IgM clones (r=0.64, p=0.011) (FIGS. 2B, 2H). The IgG titer also correlated with the frequencies of IgA+CD27+ B cells (r=0.75, p=0.013) and IgD-CD27+ memory B cells (r=0.69, p=0.0048) (FIGS. 2b, 2i, 2j) These correlations were not observed in the patients who did not need IgG-RT (FIG. 2A).

### V and J Gene Diversity

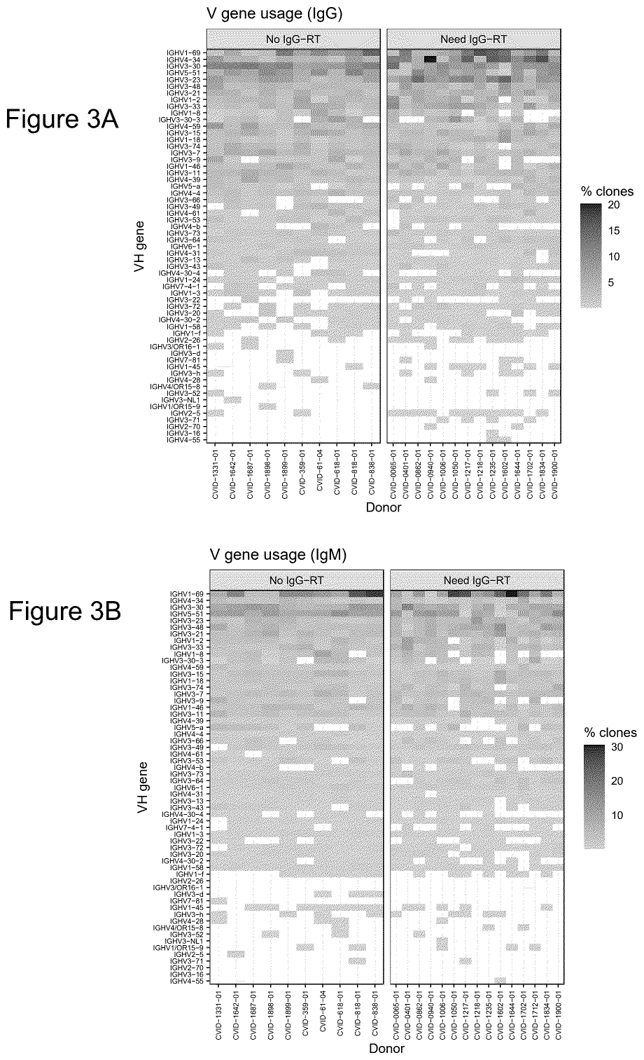

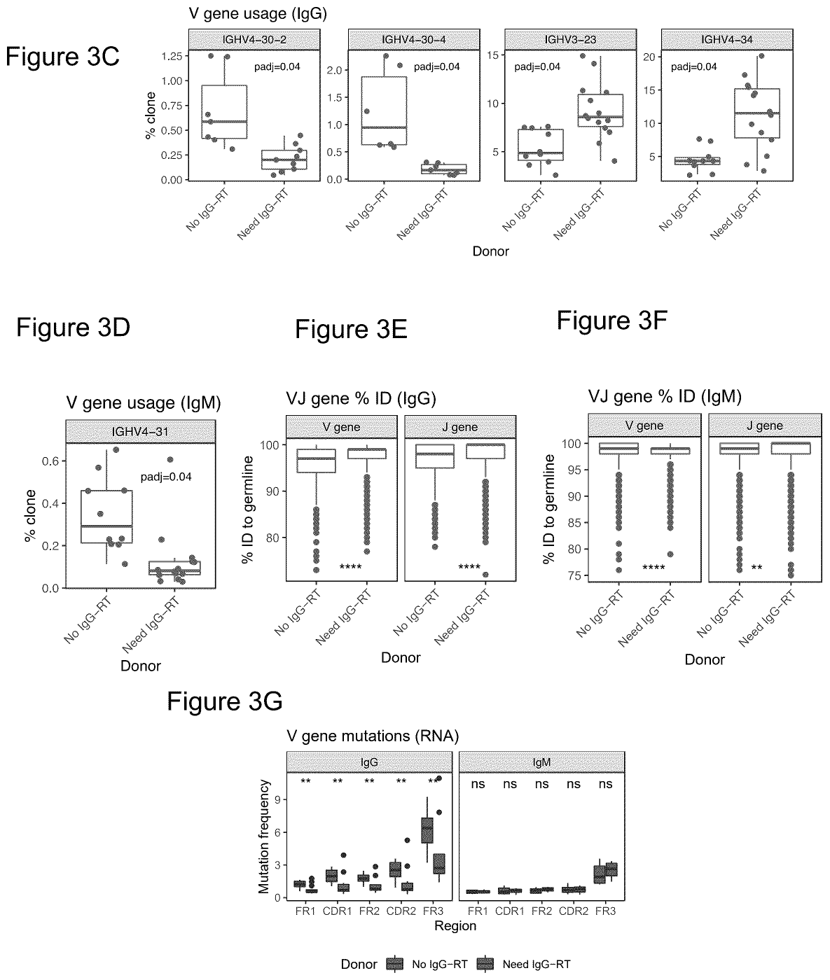

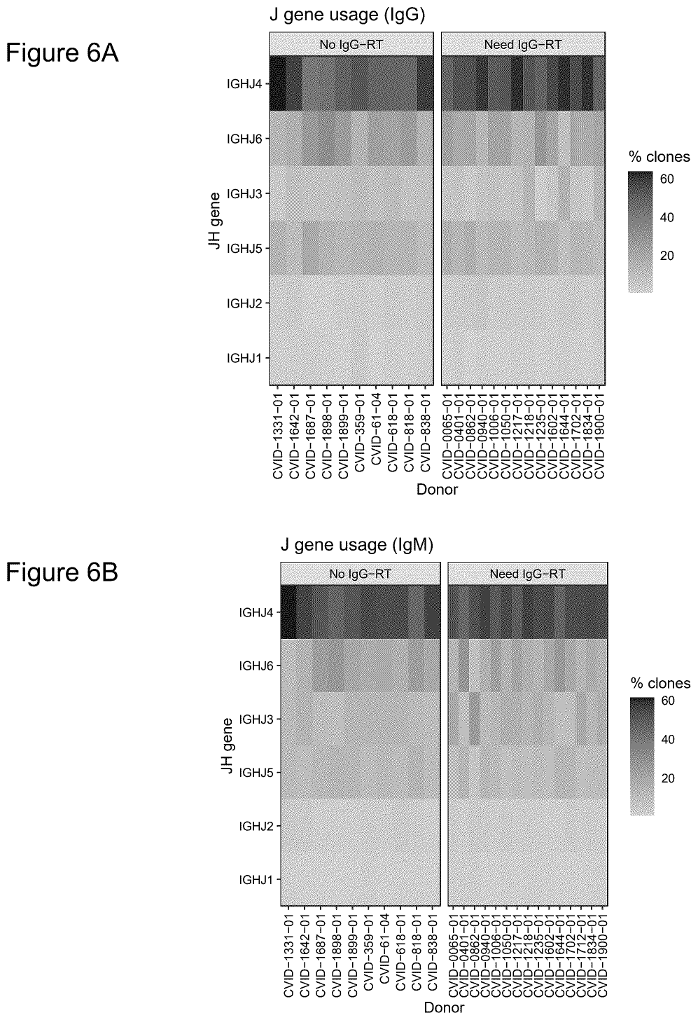

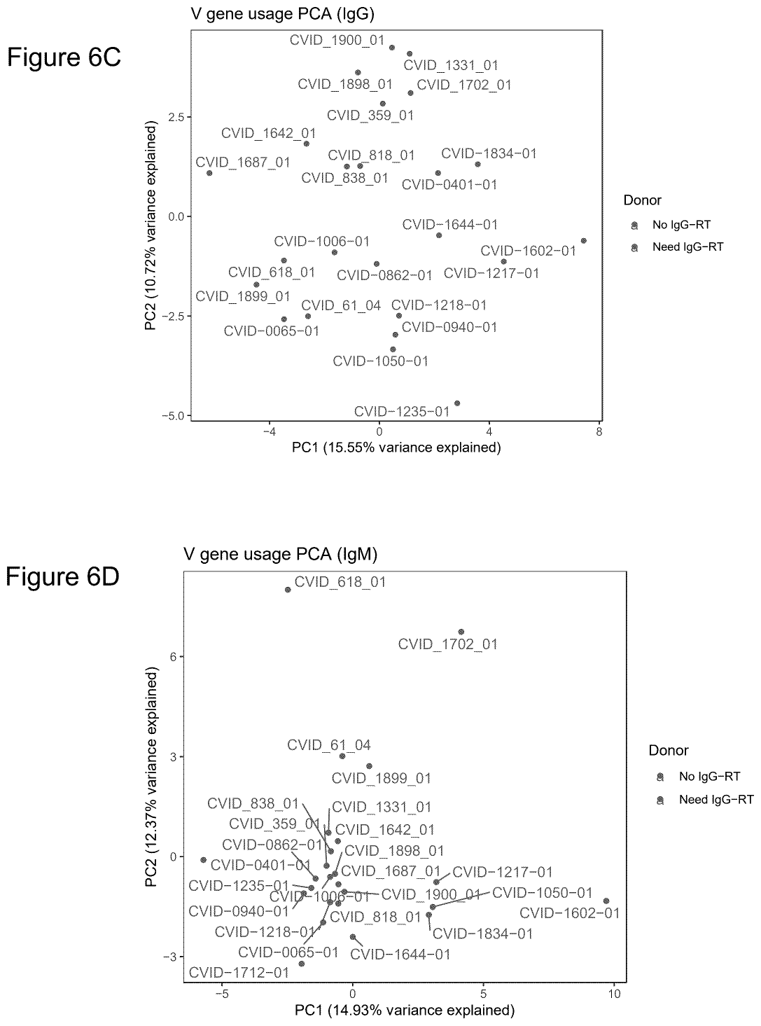

V (D) J (variable, diversity, joining) recombination, which assembles antibody gene segments during B cell development, contributes to the vast combinatorial diversity of antibodies. Applicant evaluated whether V (D) J diversity differs between patients who did and did not need IgG-RT. For IgG and IgM, both patient cohorts displayed diverse V and J gene usage (FIGS. 3A, 3B, 6A, 6B). Interestingly, principal component analysis (PCA) of the IgG V gene frequencies revealed that the patients clustered based on their need for IgG-RT. Principal component 1 (PC1), which explained 15.55% of the variance in V gene usage frequencies, separated the patients who did and did not need IgG-RT (FIG. 6C). PCA of IgM V gene usage frequencies showed clustering of the patient cohorts to a lesser extent (FIG. 6D). Next, we compared V gene frequencies between patients who did and did not need IgG-RT. Compared to the patients who did not need IgG-RT, those who needed IgG-RT had fewer IgG antibody clones with the IGHV4-30-2 and IGHV4-30-4 heavy chain V genes (Benjamini-Hochberg adjusted p-values=0.04) (FIG. 3C). The patients who needed IgG-RT also had elevated numbers of antibody clones with the IGHV3-23 and IGHV4-34 V genes (adjusted p=0.04) (FIG. 3c). While the patients who did not need IgG-RT had on average 4.53% of IGHV4-34 clones, consistent with the gene's 3-9% prevalence in adult B lymphocytes, the patients who needed IgG-RT had on average 11.27% of IGHV4-34 antibody clones (FIG. 3C). Notably, antibodies with the IGHV4-34 V gene have been shown to be self-reactive and are more common in naïve B cell repertoire than in memory B cells. We also examined differences in IgM V gene usage. The patients who needed IgG-RT had fewer IgM clones with the IGHV4-31 V gene (adjusted p=0.04) (FIG. 3D). Finally, we examined J gene usage frequencies and did not observe any significant difference between the patient cohorts for either IgG or IgM.

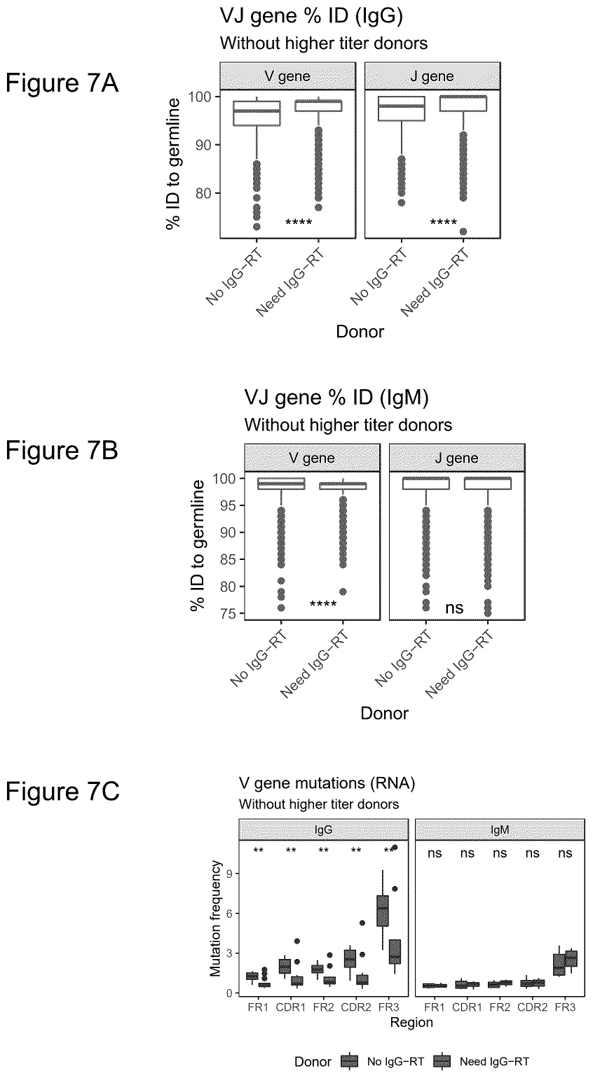

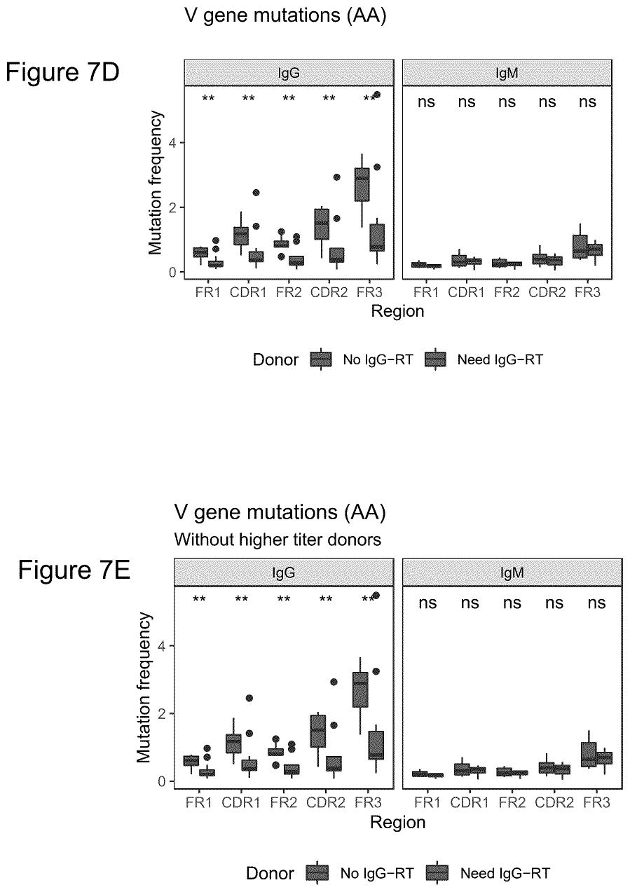

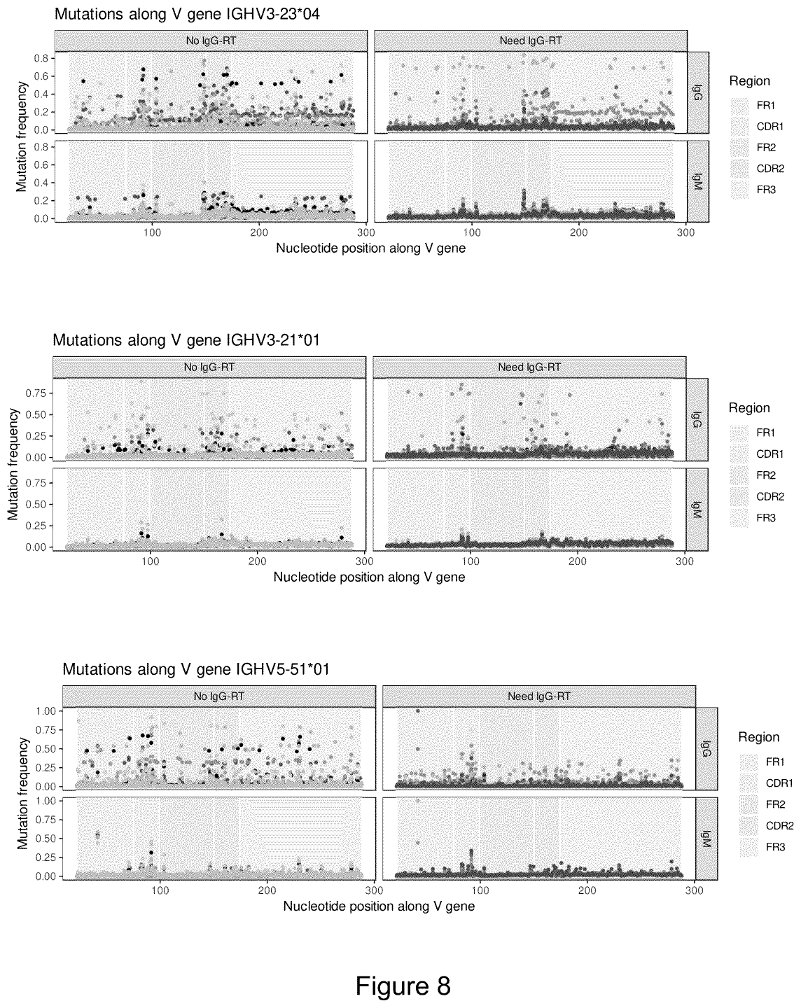

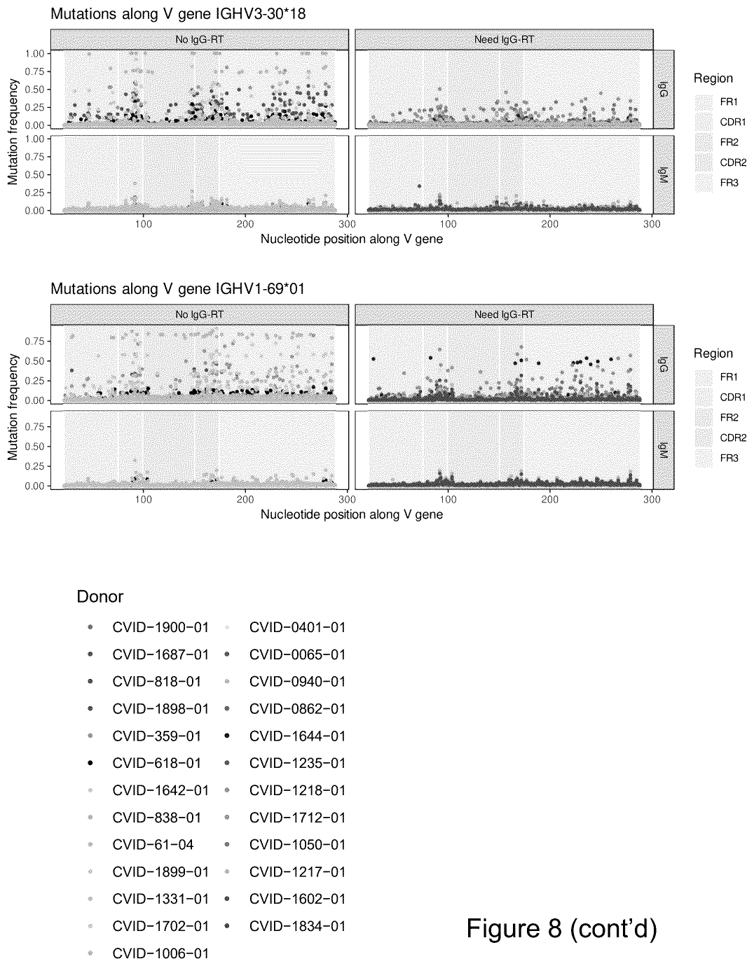

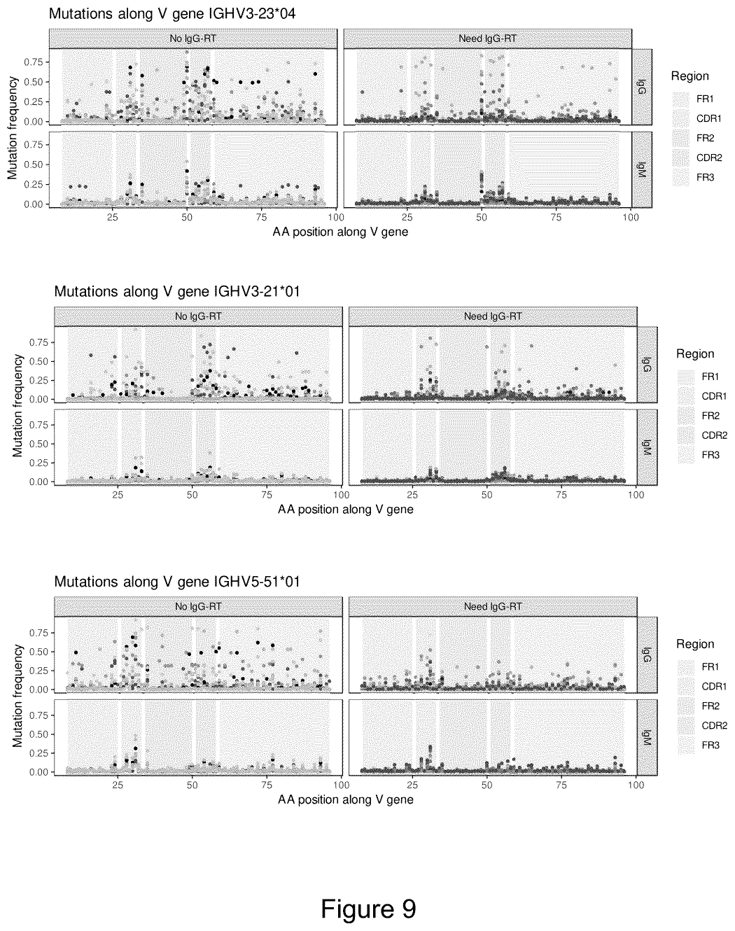

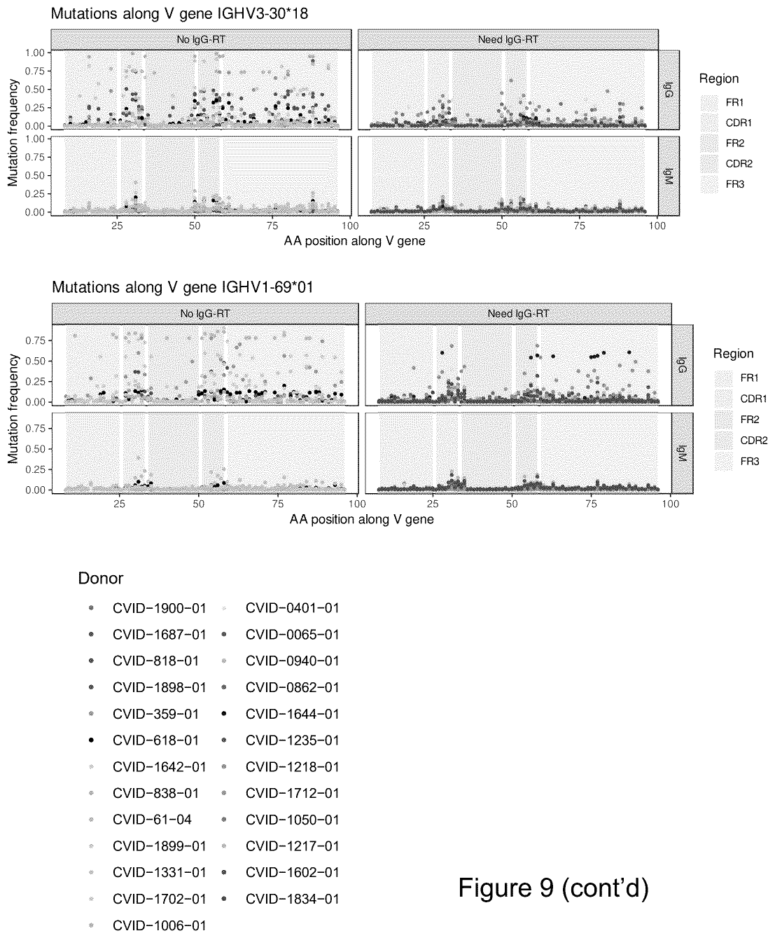

Somatic hypermutation, the process in which point mutations accumulate across the antibody V (D) J regions, further contributes to antibody diversity. Somatic hypermutation is also an important means for generating high affinity antibodies. We measured the nucleotide percent identity of the antibody heavy chain V and J gene to their respective germline sequences. Specifically, to identify germline V and J gene identity, antibody nucleotide sequences were mapped to human V and J gene reference sequences (release 202243-1, 24 Oct. 2022) from IMGT, using UBLAST. The UBLAST alignment with the lowest E-value was used to assign V and J gene families and compute percent identity to germline sequences. Interestingly, compared to patients who did not need IgG-RT, those who needed IgG-RT had significantly higher IgG V and J gene percent germline identity (p≤0.0001; FIG. 3E). For IgM, although the V gene percent germline identity was significantly lower for those who needed IgG-RT (p≤0.0001: FIG. 3F), the average difference was minor (98.15% for No IgG-RT versus 98.39% for Need IgG-RT). The differences in IgG V and J gene percent germline identities remained significant when the donors with the higher IgG/IgM titer were removed from the dataset, suggesting that the observation was not driven by the highest titer donors (FIGS. 7A, 7B). To further investigate the difference in V gene mutations between the two patient cohorts, we measured mutation frequencies in different regions along V genes, including the framework regions (FR1, FR2, FR3) and the complementarity determining regions (CDR1, CDR2). The patient cohort who needed IgG-RT had significantly (p≤0.05) lower mutation frequencies across all V gene regions, at both the nucleotide level (FIGS. 3G, 7C) and the deduced protein level (FIGS. 7D, 7E), for IgG but not for IgM. Visualizations of mutation frequencies along the most common V genes further illustrated the lower IgG V gene mutation rates in patients who needed IgG-RT (FIGS. 8, 9).

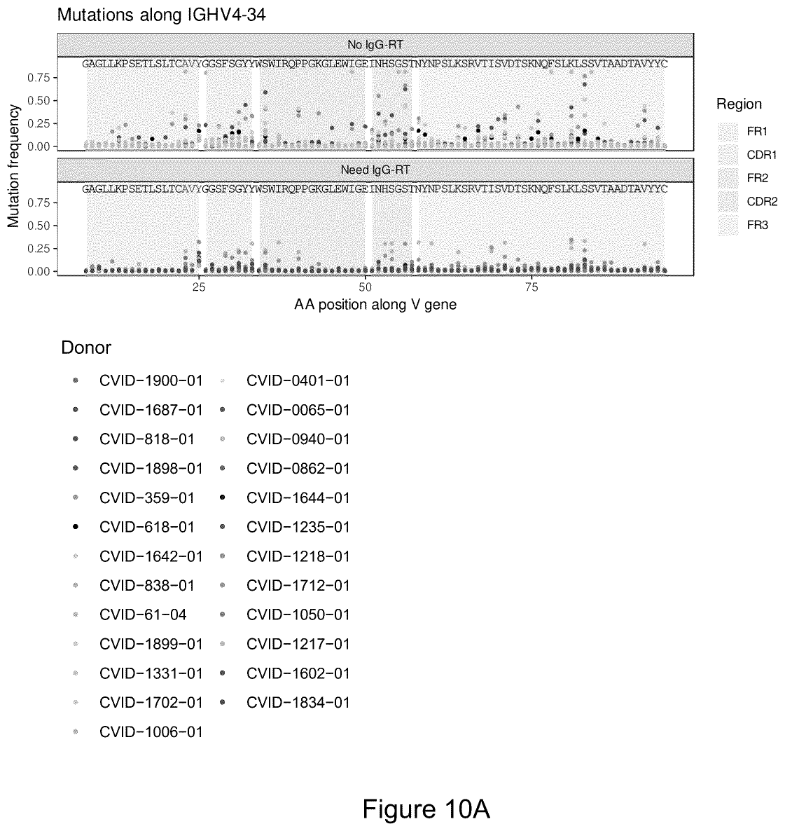

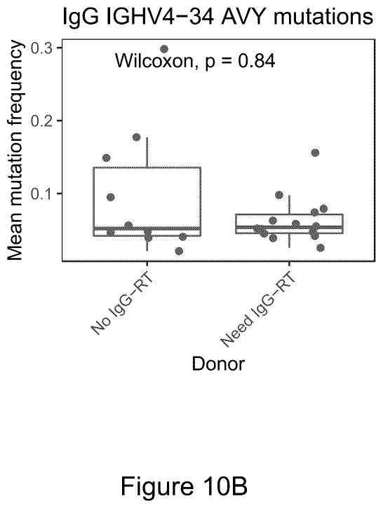

Finally, we measured the frequencies of somatic hypermutation along V gene IGHV4-34 that had elevated usage in IgG for donors who needed IgG-RT. Compared to patients who did not need IgG-RT, patients who needed IgG-RT had lower somatic hypermutations along IGHV4-34 (FIG. 10A). Previous studies indicated that the self-reactivity of IGHV4-34 antibodies is mediated by a hydrophobic patch in the framework I region, and that somatic hypermutation in the region can remove self-reactivity. However, there was no significant difference in mutation frequency in the hydrophobic patch (AVY residues) when comparing the two cohorts (FIG. 10B).

Overall, these data show that IgG hypogammaglobulinemia patients who did and did not need IgG-RT had antibody repertoires with different V gene diversities. Patients who needed IgG-RT displayed higher usage of a naïve antibody repertoire-associated V gene and had less somatic hypermutation in their IgG clones, possibly suggesting less mature antibody repertoires leaving these patients more susceptible to infection.

### CONCLUSIONS

The decision to treat hypogammaglobinemia patients with IgG-RT can be challenging, because both IgG levels and infection susceptibility vary among patients. IgG levels do not always predict a patient's infection susceptibility, and in some cases, IgG-RT is recommended for patients with asymptomatic hypogammaglobulinemia because of the potential risk of severe infections. Furthermore, both symptomatic and asymptomatic hypogammaglobinemia patients can respond well to tetanus vaccines, while diphtheria response is often impaired. Indeed, most patients in this study had a positive response to tetanus vaccine, before IgG-RT started for those who need it, while many did not respond to diphtheria (Table 1). In addition, 8 of 9 patients who did not need IgG-RT that were vaccinated with pneumococcal polysaccharides had a positive response, while only 1 of 4 patients who needed IgG-RT responded.

Hypogammaglobulinemia patients who did and did not need IgG-RT had multiple differences in their peripheral B cell receptor repertoires. Patients who needed IgG-RT had more IgG antibody clones, a higher IgM diversity index, and less oligoclonal IgG and IgM repertoires. Their IgG clones displayed distinct heavy chain V gene usage, had higher frequencies of sequences with a naïve B cell repertoire-associated V gene, and their IgG clones had less somatic hypermutation and looked more similar to germline sequences. The lower level of clonal antibody expansion and somatic hypermutation suggests that these infection susceptible patients have relatively immature B cell receptor repertoires that may be less effective against pathogens. A reduced frequency of somatic hypermutation was found in the B cell receptor repertoire of common variable immunodeficiency (CVID) patients as well, further suggesting impaired repertoire specification in the germinal centers. Interestingly, the patients in need of IgG-RT showed increased IGHV4-34 and IGHV3-23 gene usage compared to the patients without the need of IgG-RT. The IGHV4-34 increase was observed in CD19-deficient patients, patients with Wiskott-Aldrich syndrome (WAS), and RAG deficiency patients, indicating its role in self-reactive autoantibodies. Tipton et al. summarized reports of increased IGHV4-34 gene usage in systemic lupus erythematosus patients, concluding another hallmark in the repertoire of the disease, defective tolerance and 9G4-idiotype autoantibodies. The IGHV3-23 gene has been shown to be associated with the exposure to self and/or environmental antigens and is relatively abundant in humans. IGHV3-23 gene usage was also reported in hairy cell leukemia, diffuse large B-cell lymphoma, after the immunization of malaria-naïve individuals with PfSPZ-CVac, HIV patients, and in CD21 (low) B cells from WAS patients.

Conversely, hypogammaglobulinemia patients who did not need IgG-RT had relatively expanded and antigen-experienced B cell repertoires that appear to be adapted to better overcome infection susceptibility. These patients revealed elevated gene usage of IGHV4-30-2, IGHV4-30-4, and IGHV4-31 compared to the patients in need of IgG-RT. An increase of IGHV4-30-2 and -4 has been reported in WAS patients as well, demonstrating abnormalities of immune repertoire in both cohorts.

This study shows that peripheral B cell receptor sequencing can be utilized in the decision-making process for or against the use of IgG-RT in the setting of hypogammaglobulinemia.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 1
A summary of clinical parameters and immune cell phenotyping.

| | | | | | | | | | | | IgD− |
|---|---|---|---|---|---|---|---|---|---|---|---|
| | Need | | | | # of | # of | | | CD19+ | IgA+ | CD27+ |
| | IgG- | Titer | Titer | Titer | clones | clones | Diversity | Diversity | B cells/ | CD27+ | memory |
| Donor ID | RT? | (IgG) | (IgM) | (IgA) | (IgG) | (IgM) | (IgG) | (IgM) | μL | (%) | (%) |
| CVID-1331-01 | No | 4.18 | 0.17 | 0.06 | 746 | 2618 | 250.7 | 540.8 | 930 | 0.01 | 0.71 |
| CVID-1642-01 | No | 3.07 | 1.2 | 1.78 | 464 | 2413 | 17.8 | 367.3 | 89 | 1.14 | 3.89 |
| CVID-1687-01 | No | 0.96 | 0.18 | 1.39 | 160 | 3434 | 28.3 | 91.0 | 38 | 2.42 | 3.22 |
| CVID-1898-01 | No | 1.67 | 0.33 | 0.43 | 500 | 3143 | 11.2 | 155.6 | 60 | NA | 10.09 |
| CVID-1899-01 | No | 3.77 | 0.38 | 1.64 | 161 | 1936 | 3.8 | 35.4 | 98 | 7.34 | 20.38 |
| CVID-359-01 | No | 1.81 | 0.46 | 0.62 | 572 | 2391 | 28.9 | 166.2 | 454 | 0.37 | 2.35 |
| CVID-61-04 | No | 3.51 | 0.19 | 0.42 | 310 | 3482 | 14.1 | 161.4 | 87 | 0.8 | 2.08 |
| CVID-618-01 | No | 3.02 | 0.17 | <0.06 | 911 | 1839 | 40.3 | 102.0 | 133 | 0 | 6.53 |
| CVID-818-01 | No | 1.5 | 0.52 | 0.31 | 683 | 2653 | 53.8 | 116.7 | 121 | 0.55 | 2.07 |
| CVID-838-01 | No | 3.42 | 0.28 | 0.43 | 323 | 3416 | 9.1 | 52.3 | 79 | 1.17 | 2.16 |
| CVID-0065-01 | Yes | 3.54 | 0.35 | NA | 2489 | 3263 | 555.34 | 581.51 | 186 | 1.30 | 3.81 |
| CVID-0401-01 | Yes | 0.96 | 0.19 | 0.06 | 659 | 2391 | 18.45 | 825.04 | 302 | 0.00 | 0.81 |
| CVID-0862-01 | Yes | 1.44 | NA | 0.06 | 674 | 2447 | 26.48 | 278.98 | 294 | 0.01 | 1.89 |
| CVID-0940-01 | Yes | 1.42 | 0.18 | 0.06 | 934 | 2987 | 27.79 | 251.09 | 65 | 0.00 | 3.91 |
| CVID-1006-01 | Yes | 3.46 | 0.10 | 0.10 | 671 | 3373 | 6.73 | 166.45 | 294 | NA | 9.68 |
| CVID-1050-01 | Yes | 0.65 | 0.08 | 0.06 | 1710 | 2448 | 259.50 | 1097.57 | 128 | NA | 0.61 |
| CVID-1217-01 | Yes | 0.80 | 0.25 | 0.06 | 2500 | 2907 | 99.26 | 1239.84 | 477 | 0.07 | 1.32 |
| CVID-1218-01 | Yes | 4.00 | 0.60 | 0.30 | 1297 | 3062 | 45.72 | 1209.50 | 38 | 0.68 | 3.44 |
| CVID-1235-01 | Yes | 3.93 | 0.20 | 0.16 | 1376 | 3001 | 43.50 | 437.46 | 193 | 2.62 | 6.45 |
| CVID-1602-01 | Yes | 0.60 | 0.20 | 0.06 | 2189 | 2203 | 1153.95 | 903.56 | 535 | 0.00 | 0.62 |
| CVID-1644-01 | Yes | 1.55 | 0.09 | 0.06 | 1207 | 3106 | 71.94 | 938.52 | 61 | NA | 0.43 |
| CVID-1702-01 | Yes | 0.97 | 0.18 | 0.09 | 1378 | 3028 | 397.02 | 1516.81 | 172 | 0.05 | 0.47 |
| CVID-1712-01 | Yes | 0.83 | NA | 0.06 | NA | 2574 | NA | 1214.25 | 237 | NA | 0.24 |
| CVID-1834-01 | Yes | 3.00 | 0.30 | 0.20 | 597 | 3514 | 17.73 | 344.20 | 82 | 0.08 | 0.66 |
| CVID-1900-01 | Yes | 0.81 | 0.45 | 0.06 | 425 | 2798 | 153.0 | 543.1 | 256 | NA | 2.5 |

| | | | | | | | | On IgG | |
|---|---|---|---|---|---|---|---|---|---|
| | | | | | | | | replacement | |
| | IgD+ | | | | | | | at the time | Duration |
| | CD27− | | | | | | | of the blood | and |
| | naive B | IgD+ | IgG+ | | | | | draw for RNA | frequency |
| | cells | CD27+/ | CD27+ | | Vaccination | Non-infectious | Infection | isolation and | of IgG |
| Donor ID | (%) | μL | (%) | Sex | responses | diagnoses | history | sequencing? | replacement |
| CVID- | 18.15 | 480.25 | 0.13 | Male | Tetanus and | Splenomegaly, dust | Mild upper | No | Not |
| 1331-01 | | | | | diphtheria | mite allery suspected | respatory tract | | applicable |
| | | | | | positive, PnPS | | infections, not | | |
| | | | | | not tested | | treated by IgG- | | |
| | | | | | | | RT | | |
| CVID- | 83.24 | 5.07 | 0.86 | Male | Tetanus and | Crohn's disease, | No infections | No | Not |
| 1642-01 | | | | | diphtheria | hepatosplenomegaly | over last year | | applicable |
| | | | | | positive | | without IgG-RT | | |
| | | | | | PnPS limited | | | | |
| | | | | | with 23 mg/l | | | | |
| | | | | | (Prevenar | | | | |
| | | | | | vaccination | | | | |
| | | | | | February 2017) | | | | |
| CVID- | 84.39 | 0.76 | 0.04 | Male | Diphtheria | Nephrotic syndrome, | None | No | Not |
| 1687-01 | | | | | negative, | autoimmune origin | | | applicable |
| | | | | | tetanus positive, | suspected; secondary | | | |
| | | | | | lack of PnPS | hypogammaglobulinemia | | | |
| | | | | | response | possible | | | |
| CVID- | 70.58 | 9.83 | NA | Female | Diphtheria toxin IgG | Secondary | None | No | Not |
| 1898-01 | | | | | ELISA [IU/ml] | hypogammaglobulinemia | | | applicable |
| | | | | | 0.11/positive; | possible (enteral loss), | | | |
| | | | | | tetanus toxoid | hypoalbuminemia | | | |
| | | | | | IgG-ELISA | | | | |
| | | | | | [IU/ml] 1.17/ | | | | |
| | | | | | positive; Test | | | | |
| | | | | | [unit] 2022 Apr. 14 | | | | |
| | | | | | 2026 May 14 Ref. | | | | |
| | | | | | range; Pneumococcal | | | | |
| | | | | | polysaccharide | | | | |
| | | | | | (23-valent) | | | | |
| | | | | | antibody (IgG) | | | | |
| | | | | | [mg/L] 7.35 | | | | |
| | | | | | 85.79, 10-191; | | | | |
| | | | | | summary: Evidence | | | | |
| | | | | | of vaccine response | | | | |
| | | | | | to Pneumovax | | | | |
| CVID- | 49.44 | 16.23 | 4.57 | Male | PnPS positive | Recurrent prostatitis, | No | No | Not |
| 1899-01 | | | | | | suspected celiacs | infections | | applicable |
| | | | | | | disease | over last | | |
| | | | | | | | year | | |
| | | | | | | | without | | |
| | | | | | | | IgG-RT | | |
| CVID- | 83.32 | 13.71 | 0.8 | Female | Tetanus and | Thyroid disease | No | No | Not |
| 359-01 | | | | | diphtheria positive, | | respiratory | | applicable |
| | | | | | EBV seropositive, | | infections, | | |
| | | | | | Varicella positive, | | had vaginal | | |
| | | | | | PnPS: partial | | candidias | | |
| | | | | | vaccination response | | is and HP- | | |
| | | | | | (2-fold increase | | associated | | |
| | | | | | IgG for 4 of 8 | | gastritis | | |
| | | | | | serotypes) | | | | |
| CVID- | 81.66 | 7.58 | 0.69 | Male | Tetanus positive, | Splenomegaly | No | No | Not |
| 61-04 | | | | | diphtheria positive, | | infections | | applicable |
| | | | | | pertussis negative, | | over last | | |
| | | | | | HBV positive after | | years | | |
| | | | | | repeated vaccination, | | without | | |
| | | | | | predominantly | | IgG-RT | | |
| | | | | | low-titer IgG | | | | |
| | | | | | antibody levels | | | | |
| | | | | | against | | | | |
| | | | | | pneumococcal | | | | |
| | | | | | polysaccharides | | | | |
| CVID- | 70.84 | 19.02 | 0.74 | Male | Lack of | Suspected rosacea, | No | No | Not |
| 618-01 | | | | | vaccination | recurrent non- | infections | | applicable |
| | | | | | response to | infectious diarrhea | over last | | |
| | | | | | diphtheria, | | year | | |
| | | | | | hepatitis, TBE, | | without | | |
| | | | | | hepatitis B, and | | IgG-RT | | |
| | | | | | pneumococcal | | | | |
| | | | | | polysaccharides. | | | | |
| CVID- | 76.23 | 3.68 | 1.1 | Male | Tetanus positive, | Splenomegaly, | No | No | Not |
| 818-01 | | | | | diphtheria | pollen allergy | infections | | applicable |
| | | | | | negative, | | over last | | |
| | | | | | limited vaccine | | years | | |
| | | | | | response to | | without | | |
| | | | | | Pneumovax August | | IgG-RT | | |
| | | | | | 2007 and March 2012 | | | | |
| CVID- | 83.5 | 7.29 | 0.48 | Male | PnPS(23-valent) | Recurrent non- | No | No | Not |
| 838-01 | | | | | positive: PnPS- | infectious | infections | | applicable |
| | | | | | specific-IgG | diarrhea, allergic | over last | | |
| | | | | | 103.46 mg/L (norm: | rhinitis, previous | year | | |
| | | | | | 10-191) | upper respiratory | without | | |
| | | | | | | infections | IgG-RT | | |
| CVID- | 69.66 | 29.52 | 0.79 | Female | October 2011 | Rhinoconjunctivitis | Recurrent | Yes | 48 g/month |
| 0065-01 | | | | | tetanus-specific | | bronchitis | | for the last |
| | | | | | antibodies 0.96 | | | | 8 years |
| | | | | | IU/ml (borderline), | | | | |
| | | | | | after vaccination | | | | |
| | | | | | in 1998;, | | | | |
| | | | | | diphtheria | | | | |
| | | | | | negative but no | | | | |
| | | | | | booster documented; | | | | |
| | | | | | HBs-antibody | | | | |
| | | | | | titer >1,000 U/l; | | | | |
| | | | | | PnPS vaccination | | | | |
| | | | | | 2027 Oct. 11, but | | | | |
| | | | | | no testing of | | | | |
| | | | | | vaccination | | | | |
| | | | | | response | | | | |
| CVID- | 65.02 | 18.63 | 0.27 | Female | Diphtheria | Rheumatoid arthritis | Sinus | Yes | 20 g/month |
| 0401-01 | | | | | negative, | | infections, | | for the last |
| | | | | | tetanus positive, | | recurrent | | 22 years |
| | | | | | PnPS not | | bronchitis, | | |
| | | | | | vaccinated | | multiple | | |
| | | | | | | | pneumonia | | |
| CVID- | 80.39 | 10.23 | 0.48 | Male | None documented | Lymphadenopathy, | Respiratory | Yes | 50 g/month |
| 0862-01 | | | | | | enteropathy | tract | | for the last |
| | | | | | | | | | 4 years |
| CVID- | 70.58 | 10.98 | 0.69 | Male | No response to | Mild splenomegaly | Upper | Yes | 32 g/month |
| 0940-01 | | | | | PnPS vaccination | | respiratory | | for the last |
| | | | | | | | tract | | year |
| CVID- | 46.57 | 116.84 | NA | Female | Tetanus positive, | Hypothyroid goiter | Pneumonia | Yes | 13.3 g/month |
| 1006-01 | | | | | lack of | | | | for the last |
| | | | | | vaccination | | | | year |
| | | | | | response to | | | | |
| | | | | | diphtheria and | | | | |
| | | | | | PnPS | | | | |
| CVID- | 88.64 | 9.18 | NA | Female | Tetanus negative, | GLILD | Recurrent | Yes | 40 g/month |
| 1050-01 | | | | | diphtheria | | otitis | | for the last |
| | | | | | negative, PnPS | | and sinus | | 4 months |
| | | | | | not tested | | infections, | | |
| | | | | | | | recurrent | | |
| | | | | | | | bronchitis, | | |
| | | | | | | | multiple | | |
| | | | | | | | pneumonia | | |
| CVID- | 32.06 | 205.06 | 0.22 | Female | None documented | ITP | Recurrent | Yes | 30 g/month |
| 1217-01 | | | | | | | bronchitis, | | for the last |
| | | | | | | | pneumonia, | | 3 years |
| | | | | | | | meningitis | | |
| CVID- | 69.68 | 6.41 | 0.49 | Female | None documented | GLILD, autoimmune | Upper | Yes | 160 g/month |
| 1218-01 | | | | | | enteropathy | respiratory | | for the last |
| | | | | | | | tract | | 4 years |
| CVID- | 84.62 | 7.06 | 2.39 | Female | None documented | Autoimmune thyroiditis | Upper | Yes | 10 g/month |
| 1235-01 | | | | | | | respiratory | | for the last |
| | | | | | | | tract | | 6 years |
| CVID- | 31.98 | 176.23 | 0.12 | Male | Medium- to | GLILD | Pneumonia | Yes | 30 g/month |
| 1602-01 | | | | | low-titer IgG | | | | for the last |
| | | | | | antibodies to | | | | 6 months |
| | | | | | pneumococcal | | | | |
| | | | | | polysaccharides | | | | |
| CVID- | 91.95 | 3.86 | NA | Female | Lack of | GLILD | Pneumonia | Yes | 26.7 g/month |
| 1644-01 | | | | | vaccination | | | | for the last |
| | | | | | protection | | | | 2 months |
| | | | | | against tetanus | | | | |
| | | | | | and diphtheria | | | | |
| | | | | | in June 2014 (but | | | | |
| | | | | | vaccination >10 | | | | |
| | | | | | yrs ago); testing | | | | |
| | | | | | PnPS vaccination | | | | |
| | | | | | response not | | | | |
| | | | | | done due to | | | | |
| | | | | | urgent need for | | | | |
| | | | | | IgG-RT | | | | |
| CVID- | 59.79 | 17.84 | 0.14 | Female | Lack of IgG | GLILD | Upper | No, but | Not |
| 1702-01 | | | | | antibody levels | | respiratory | IgG-RT | applicable |
| | | | | | against | | tract | started | |
| | | | | | pneumococcal | | | after the | |
| | | | | | polysaccharides | | | blood draw | |
| CVID- | 94.44 | 10.50 | NA | Female | None documented | Lymphadenopathy, | Sinus | Yes | 30 g/month |
| 1712-01 | | | | | | enteropathy, microcytic | infections, | | for the last |
| | | | | | | anemia, vitamin D | recurrent | | 2 years |
| | | | | | | deficiency | bronchitis | | |
| CVID- | 52.38 | 3.30 | 0.17 | Female | Tetanus- | ITP, GLILD | Upper | Yes | 30 g/month |
| 1834-01 | | | | | specific IgG | | respiratory | | for the last |
| | | | | | April 2018 | | tract | | year |
| | | | | | positive, | | | | |
| | | | | | diphtheria | | | | |
| | | | | | April 2018 | | | | |
| | | | | | negative | | | | |
| | | | | | (vaccination in | | | | |
| | | | | | 2007), PnPS | | | | |
| | | | | | not examined | | | | |
| CVID- | 67.9 | 71.94 | NA | Female | Diphtheria | Hashimoto thyreoiditis | Recurrent | No, but | Not |
| 1900-01 | | | | | negative, | | respiratory | IgG-RT | applicable |
| | | | | | tetanus positive, | | infections, | started | |
| | | | | | hepatitis B | | otitis media, | after the | |
| | | | | | positive, | | purulent | blood | |
| | | | | | isohemagglutinin | | conjunctivitis | draw | |
| | | | | | positive, anti- | | and herpes | | |
| | | | | | EBNA positive | | labialis | | |

NA, data not available.

TABLE 2
Table 2. The same IgG/IgM variable region forward primer pool was used to amplify
IgG or IgM (performed in separate reactions with the respective reverse primer).
The index sequence region for primers to create Illumina sequencing libraries is
indicated by XXXXXX (any Illumina index sequence can be used here).
Regions with multiple Ns are used to improve the quality of Illumina
sequening cluster generation and base calling.

| Primer description | Primer sequence | SEQ ID NO |
|---|---|---|
| For amplifying | | |
| Illumina libraries | | |
| IgG/IgM variable region | AATGATACGGCGACCACCGAGATCTACACGTT | 1 |
| forward pool: | GTAAAACGACGGCCAGT NNNNNNN | |
| | CAGRTGCAGCTGGTGCART | |
| | AATGATACGGCGACCACCGAGATCTACACGTT | 2 |
| | GTAAAACGACGGCCAGT NNNNNNN | |
| | SAGGTCCAGCTGGTRCAGT | |
| | AATGATACGGCGACCACCGAGATCTACACGTT | 3 |
| | GTAAAACGACGGCCAGT NNNNNNN | |
| | CAGRTCACCTTGAAGGAGT | |
| | AATGATACGGCGACCACCGAGATCTACACGTT | 4 |
| | GTAAAACGACGGCCAGT NNNNNNN | |
| | SAGGTGCAGCTGGTGGAG | |
| | AATGATACGGCGACCACCGAGATCTACACGTT | 5 |
| | GTAAAACGACGGCCAGT NNNNNNN | |
| | CAGGTGCAGCTACAGCAGT | |
| | AATGATACGGCGACCACCGAGATCTACACGTT | 6 |
| | GTAAAACGACGGCCAGT NNNNNNN | |
| | CAGSTGCAGCTGCAGGAGT | |
| | AATGATACGGCGACCACCGAGATCTACACGTT | 7 |
| | GTAAAACGACGGCCAGT NNNNNNN | |
| | GARGTGCAGCTGGTGCAGT | |
| | AATGATACGGCGACCACCGAGATCTACACGTT | 8 |
| | GTAAAACGACGGCCAGT NNNNNNN | |
| | CAGGTACAGCTGCAGCAGTC | |
| IgG constant region | CAAGCAGAAGACGGCATACGAGAT XXXXXX | 9 & 10 |
| reverse: | AGTCAGTCAGCCGACSGATGGGCCCTTGGTGG | |
| IgM constant region | CAAGCAGAAGACGGCATACGAGAT XXXXXX | 9 & 11 |
| reverse: | AGTCAGTCAGCCGGAATTCTCACAGGAGACGAGG | |
| For sequencing IgG | | |
| Illumina libraries | | |
| Read 1: | GACCACCGAGATCTACACGTTGTAAAACGACG | 12 |
| | GCCAGT | |
| Read 1 (for PhiX | ACACTCTTTCCCTACACGACGCTCTTCCGATCT | 13 |
| control): | | |
| Index: | CCACCAAGGGCCCATCSGTCGGCTGACTGACT | 14 |
| Read 2: | AGTCAGTCAGCCGACSGATGGGCCCTTGGTGG | 10 |
| Read 2 (for PhiX | CGGTCTCGGCATTCCTGCTGAACCGCTCTTCCG | 15 |
| control): | ATCT | |
| For sequencing IgM | | |
| Illumina libraries | | |
| Read 1: | GACCACCGAGATCTACACGTTGTAAAACGACG | 12 |
| | GCCAGT | |
| Read 1 (for PhiX | ACACTCTTTCCCTACACGACGCTCTTCCGATCT | 13 |
| control): | | |
| Index: | CCTCGTCTCCTGTGAGAATTCCGGCTGACTGACT | 16 |
| Read 2: | ACGAGGGGGAAAAGGGTTGGGGCGGATGCAC | 17 |
| Read 2 (for PhiX | CGGTCTCGGCATTCCTGCTGAACCGCTCTTCCG | 15 |
| control): | ATCT | |

What is claimed is:

1. A method of selecting a hypogammaglobulinemia patient for immunoglobulin replacement therapy (IgG-RT), comprising
(1) obtaining sequence information of at least 10,000 transcripts from the patient's sample comprising B cells, wherein each of the transcripts encodes a heavy chain of IgG or IgM or a portion thereof; and
(2) characterizing B cell receptor (BCR) repertoire of the patient by identifying antibody clones using the sequence information;
(3) analyzing the BCR repertoire by one or more selected from (a)-(f):
(t) measuring the number or abundance of individual antibody clones in the BCR repertoire;
(u) calculating a diversity index value of the antibody clones by measuring the number and abundance of individual antibody clones in the BCR repertoire;
(v) selecting at least 10 but no more than 30 antibody clones that are most frequent in the BCR repertoire and calculating a total frequency of the 10 to 30 most frequent antibody clones;
(w) determining a variable region gene (V region) usage frequency in the antibody clones, optionally wherein the V region gene is selected from the group consisting of IGHV4-30-2 heavy chain V region gene, IGHV4-30-4 heavy chain V region gene, IGHV3-23 gene, IGHV4-34 heavy chain V region gene, and IGHV4-31 heavy chain V region gene;
(x) measuring a percent germline identity by comparing the V or J region in the BCR repertoire against a corresponding germline sequence; and
(y) determining the somatic mutation frequency in different regions along the V region;
(4) selecting a patient for IgG-RT when one or more of (g)-(s) are satisfied:
(z) the number of IgG clones in the BCR repertoire is greater than Tg, wherein Tg is at least 600;
(aa) the diversity index value of the IgM antibody clones is greater than Th, wherein Th is at least 250;
(bb) the total frequency of the most frequent 10 to 30 IgG antibody clones in the BCR repertoire is less than Ti, wherein Ti is at most 30%;
(cc) the total frequency of the most frequent 10 to 30 IgM antibody clones in the BCR repertoire is less than Tj, wherein Tj is at most 7%;
(dd) the frequency of IgG antibody clones with the IGHV4-30-2 heavy chain V region is less than Tk, wherein Tk is at most 0.3%;
(ee) the frequency of IgG antibody clones with the IGHV4-30-4 heavy chain V region is less than Tl, wherein Tl is at most 0.5%;
(ff) the frequency of IgG antibody clones with the IGHV3-23 heavy chain V region is greater than Tm, wherein Tm is at least 6%;
(gg) the frequency of IgG antibody clones with the IGHV4-34 heavy chain V region is greater than Tn, wherein Tn is at least 6%;
(hh) the frequency of IgM antibody clones with the IGHV4-31 heavy chain V region is less than To, wherein To is at most 0.2%;
(ii) IgG V gene average percent germline identity is greater than Tp, wherein Tp is at least 98%;
(jj) IgG J gene average percent germline identity is greater than Tq, wherein Tq is at least 98%;
(kk) the median somatic nucleotide mutation frequency in the FR1, CDR1, FR2, or CDR2 regions of IgG is lower than Tr, wherein Tr is at most 1; and
(ll) the median somatic nucleotide mutation frequency in the FR3 region of IgG is less than Ts, wherein Ts is at most 4;
(5) providing information related to whether or not the patient needs IgG-RT.

2. The method of claim 1, wherein in step (1), obtaining sequence information of at least 50,000 transcripts.

3. The method of claim 0, wherein in step (1), obtaining sequence information of at least 100,000, 500,000, 1000,000 or 10 million transcripts.

4. The method of any one of claims 1-3, wherein in step (4), the patient for IgG-RT is selected when (g) is satisfied.

5. The method of any one of claims 1-0, wherein in (g), Tg is 600, 650, 700, 750, 800, 850, 900, 950, 1,000, 1,050, or 1,100.

6. The method of any one of claims 1-0, wherein in (g), Tg is between 700 and 1,200, between 700 and 1,100, between 700 and 1,000, between 800 and 1,100, between 800 and 1,000, between 900 and 1,100, between 900 and 1,000, between 800 and 900, or between 700 and 800.

7. The method of any one of claims 1-6, wherein in step (4), the patient for IgG-RT is selected when (h) is satisfied.

8. The method of any one of claims 1-7, wherein in (h), Th is 250, 300, 350, 400, 450, 500, 550, or 600.

9. The method of any one of claims 1-7, wherein in (h), Th is between 250 and 650, between 300 and 650, between 350 and 650, between 400 and 650, between 450 and 650, between 250 and 600, between 300 and 600, between 350 and 600, between 400 and 600, between 450 and 600, between 250 and 550, between 300 and 550, between 350 and 550, between 400 and 550, between 450 and 550, between 250 and 500, between 300 and 500, between 350 and 500, between 400 and 500, or between 450 and 500.

10. The method of any one of claims 1-9, wherein in step (4), the patient for IgG-RT is selected when (i) is satisfied.

11. The method of any one of claims 1-10, wherein in (i), Ti is 30%, 25%, or 20%.

12. The method of any one of claims 1-10, wherein in (i), Ti is between 20% and 35%, between 20% and 30%, between 20% and 25% or between 25% and 35%, or between 25% and 30%.

13. The method of any one of claims 1-12, wherein in step (4), the patient for IgG-RT is selected when (j) is satisfied.

14. The method of any one of claims 1-13, wherein in (j), Tj is 7%, 6.5%, 6%, 5.5%, 5%, 4.5%, or 4%.

15. The method of any one of claims 1-13, wherein in (j), Tj is between 4% and 7%, between 4% and 6%, between 4% and 5%, between 5% and 7%, between 5% and 6%, or between 6% and 7%.

16. The method of any one of claims 1-15, wherein in step (4), the patient for IgG-RT is selected when (k) is satisfied.

17. The method of any one of claims 1-16, wherein in (k), Tk is 0.3%, 0.25%, 0.2%, or 0.15%.

18. The method of any one of claims 1-16, wherein in (k), Tk is between 0.15% and 0.3%, between 0.2% and 0.3%, between 0.25% and 0.3%, between 0.15% and 0.25%, between 0.15% and 0.2%, between 0.2% and 0.3%, or between 0.25% and 0.3%.

19. The method of any one of claims 1-18, wherein in step (4), the patient for IgG-RT is selected when (1) is satisfied.

20. The method of any one of claims 1-19, wherein in (1), Tl is 0.5%, 0.45%, 0.4%, 0.35%, 0.3%, or 0.25%.

21. The method of any one of claims 1-19, wherein in (1), Tl is between 0.25% and 0.5%, between 0.25% and 0.45%, between 0.25% and 0.4%, between 0.25% and 0.35%, between 0.25% and 0.3%, between 0.3% and 0.5%, between 0.3% and 0.45%, between 0.3% and 0.4%, between 0.3% and 0.35%, between 0.35% and 0.45%, between 0.35% and 0.4%, between 0.4% and 0.5%, between 0.4% and 0.45%, or between 0.45% and 0.5%.

22. The method of any one of claims 1-21, wherein in step (4), the patient for IgG-RT is selected when (m) is satisfied.

23. The method of claim 1-22, wherein in (m), Tm is 6%, 7%, 8%, 9%, or 10%.

24. The method of claim 1-22, wherein in (m), Tm is between 6% and 10%, between 6% and 9%, between 6% and 8%, between 6% and 7%, between 7% and 10%, between 7% and 8%, between 8% and 10%, between 8% and 9%, or between 9% and 10%.

25. The method of any one of claims 1-24, wherein in step (4), the patient for IgG-RT is selected when (n) is satisfied.

26. The method of any one of claims 1-25, wherein in (n), Tn is 6%, 7%, 8%, 9%, or 10%.

27. The method of any one of claims 1-25, wherein in (n), Tn is between 6% and 10%, between 6% and 9%, between 6% and 8%, between 6% and 7%, between 7% and 10%, between 7% and 8%, between 8% and 10%, between 8% and 9%, or between 9% and 10%.

28. The method of any one of claims 1-27, wherein in step (4), the patient for IgG-RT is selected when (o) is satisfied.

29. The method of any one of claims 1-28, wherein in (o), To is 0.2%, 0.15%, or 0.1%.

30. The method of any one of claims 1-28, wherein in (o), To is between 0.1% and 0.2%, between 0.1% and 0.15%, or between 0.15% and 0.2%.

31. The method of any one of claims 1-30, wherein in step (4), the patient for IgG-RT is selected when (p) is satisfied.

32. The method of any one of claims 1-31, wherein in (p), Tp is 98%, 98.5% or 99%.

33. The method of any one of claims 1-31, wherein in (p), Tp is between 98% and 99%, between 98% and 98.5%, or between 98.5% and 99%.

34. The method of any one of claims 1-33, wherein in step (4), the patient for IgG-RT is selected when (q) is satisfied.

35. The method of any one of claims 1-34, wherein in (q), Tq is 98%, 98.5% or 99%.

36. The method of any one of claims 1-34, wherein in (q), Tq is between 98% and 99%, between 98% and 98.5%, or between 98.5% and 99%.

37. The method of any one of claims 1-36, wherein in step (4), the patient for IgG-RT is selected when (r) is satisfied.

38. The method of any one of claims 1-37, wherein in (r), Tris 1, 0.8, or 0.6.

39. The method of any one of claims 1-37, wherein in (r), Tr is between 0.6 and 1, between 0.6 and 0.8, or between 0.8 and 1.

40. The method of any one of claims 1-39, wherein in step (4), the patient for IgG-RT is selected when(s) is satisfied.

41. The method of any one of claims 1-40, wherein in(s), Ts is 4, 3.5, 3, 2.5, or 2.

42. The method of any one of claims 1-40, wherein in(s), Ts is between 2 and 4, between 2 and 3.5, between 2 and 3, between 2 and 2.5, between 2.5 and 4, between 2.5 and 3.5, between 2.5 and 3, between 3 and 4, between 3 and 3.5, or between 3.5 and 4.

43. The method of any one of claims 1-42, wherein in step (3), one, two, three, four, five, or six analysis out of (a) to (f) are performed for analysis of BCR repertoire.

44. The method of any one of claims 1-42, wherein in step (4), the patient for IgG-RT is selected when one, two, three, four, five, six, seven, eight, nine, ten, eleven, twelve, or thirteen criteria selected from (g) to (s) are satisfied.

45. The method of any one of claims 1-42, wherein in step (4), the patient for IgG-RT is selected when at least two, at least three, at least four, at least five, at least six, at least seven, at least eight, at least nine, at least ten, at least eleven criteria, or at least twelve criteria selected from (g) to (s) are satisfied.

46. The method of any one of claims 1-45, wherein the patient has been selected for having less than 5 g/L of serum IgG and more than 40/μL of peripheral B cells.

47. The method of claims 1-45, wherein the patient has been selected for having less than 4.5 g/L of serum IgG and more than 35/μL of peripheral B cells.

48. The method of claims 1-45, wherein the patient has been selected for having less than 4 g/L of serum IgG and more than 30/μL of peripheral B cells.

49. The method of any one of claims 1-48, wherein the patient's sample comprises peripheral blood mononuclear cells (PBMCs).

50. The method of any one of claims 1-49, further comprising the step of sequencing the at least 10,000 transcripts, thereby providing the sequence information.

51. The method of any one of claims 1-50, further comprising the step of treating the patient with IgG-RT when the patient is selected for IgG-RT.

52. A method of treating a hypogammaglobulinemia patient, comprising administering immunoglobulin replacement therapy (IgG-RT) to the patient, wherein the patient has been selected for IgG-RT using the method of any one of claims 1-51.

53. The method of treating a hypogammaglobulinemia patient, further comprising selecting the patient for IgG-RT using the method of any one of claims 1-51.

54. A diagnostic product for selecting a hypogammaglobulinemia patient for treatment with immunoglobulin replacement therapy (IgG-RT), wherein the diagnostic product is stored on a non-transitory computer readable medium and is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) characterizing B cell receptor (BCR) repertoire of each of the patients by identifying antibody clones based on the sequence information;
(3) obtaining a training dataset including a plurality of training examples, wherein each training example corresponds to a BCR repertoire of an individual patient and comprises:
(c) one or more properties related to the BCR repertoire of the individual patient; and
(d) a diagnosis of the hypogammaglobulinemia patient whether or not the individual patient needs IgG-RT, optionally, the diagnosis is based on information associated with serum IgG levels or infections susceptibility;
(4) numerically encoding the training examples in the training dataset, comprising numerically encoding the one or more properties related to the BCR repertoire of the individual patient, and numerically encoding the diagnosis of the hypogammaglobulinemia patient of whether or not the individual patient needs IgG-RT;
(5) for a diagnostic model comprising a neural network that has a plurality of layers, each layer having a plurality of parameters, the layers comprising an input layer for receiving the numerically encoded one or more properties of the BCR repertoire and an output layer indicating a likelihood of requirement of IgG-RT, for one or more iterations of the training process:
(e) for a set of training examples for the current iteration, applying parameters of the neural network to generate estimated likelihoods for the set of training examples,
(f) computing a loss function indicating a difference between the estimated likelihoods and the numerically encoded diagnoses for the set of training examples for the current iteration,
(g) repeatedly backpropagating one or more error terms obtained from the loss function to update the parameters of the layers of the diagnostic model, and
(h) stopping the backpropagation after the loss function satisfies a criterion; and
(6) storing the updated set of parameters for the layers of the diagnostic model on the computer readable storage medium.

55. The diagnostic product of claim 54, wherein in step (3) (a), the one or more properties related to the BCR repertoire of the individual patient is selected from 1) to 7),
8) the number or abundance of individual IgG clones in the BCR repertoire;
9) a diversity index value of the IgM antibody clones calculated by measuring the number and abundance of individual IgM antibody clones in the BCR repertoire;
10) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgG antibody clones that are most frequent in the BCR repertoire;
11) a total frequency of top 10, top 11, top 12, top 13, top 14, top 15, top 16, top 17, top 18, top 19, top 20, top 21, top 22, top 23, top 24, top 25, top 26, top 27, top 28, top 29, or top 30 IgM antibody clones that are most frequent in the BCR repertoire;
12) a variable region (V) gene usage frequency in the antibody clones, optionally wherein the V gene is selected from the group consisting of IGHV4-30-2 heavy chain V gene, IGHV4-30-4 heavy chain V gene, IGHV3-23 heavy chain V gene, IGHV4-34 heavy chain V gene, and IGHV4-31 heavy chain V gene;
13) an average percent germline identity measured by comparing the variable (V) or joining (J) region in the BCR repertoire against a corresponding germline sequence; and
14) a median somatic nucleotide mutation frequency in the V region in the BCR repertoire, optionally wherein the V region comprises FR1, CDR1, FR2, CDR2, and/or FR3 regions.

56. A diagnostic product for selecting a hypogammaglobulinemia patient for treatment with immunoglobulin replacement therapy (IgG-RT), wherein the diagnostic product is stored on a non-transitory computer readable medium and is manufactured by a process comprising:
(1) obtaining sequence information of a plurality of patients with hypogammaglobulinemia, wherein sequence information of each of patients comprises sequences of at least 10,000 transcripts from the patient's sample comprising B cells, wherein each of the transcripts encodes a heavy chain of IgG or IgM or a portion thereof;
(2) obtaining a training dataset including a plurality of training examples, wherein each training example comprises the sequence information of an individual patient and a diagnosis of the individual patient whether or not the individual patient needs IgG-RT, optionally, the diagnosis is based on information associated with serum IgG levels or infections susceptibility;
(3) numerically encoding the training examples in the training dataset, comprising numerically encoding the sequence information of the individual patient, and numerically encoding the diagnosis of the individual patient of whether or not the individual patient needs IgG-RT;
(4) for a diagnostic model comprising a neural network that has a plurality of layers, each layer having a plurality of parameters, the layers comprising an input layer for receiving the numerically encoded sequence information and an output layer indicating a likelihood of requirement of IgG-RT, for one or more iterations of the training process:
(e) for a set of training examples for the current iteration, applying parameters of the neural network to generate estimated likelihoods for the set of training examples,
(f) computing a loss function indicating a difference between the estimated likelihoods and the numerically encoded diagnoses for the set of training examples for the current iteration,
(g) repeatedly backpropagating one or more error terms obtained from the loss function, to update the parameters of the layers of the diagnostic model, and
(h) stopping the backpropagation after the loss function satisfies a criterion; and
(5) storing the updated set of parameters for the layers of the diagnostic model on the computer readable storage medium.

57. A method of selecting a hypogammaglobulinemia patient for immunoglobulin replacement therapy (IgG-RT), comprising
(1) obtaining sequence information of at least 10,000 transcripts from the patient's sample comprising B cells, wherein each of the transcripts encodes a heavy chain of IgG or IgM or a portion thereof;
(2) providing the sequence information or information related to B cell receptor (BCR) repertoire of the patient obtained by processing the sequence information to the diagnostic product of any one of claim 54-56 and operating the diagnostic product; and
(3) obtaining, from the diagnostic product, information related to whether or not the patient needs IgG-RT.

58. The method of claim 57, wherein the step (2) of characterizing B cell receptor (BCR) repertoire comprises analyzing the BCR repertoire by one or more steps selected from (a)-(f):
(g) measuring the number or abundance of individual antibody clones in the BCR repertoire;
(h) calculating a diversity index value of the antibody clones by measuring the number and abundance of individual antibody clones in the BCR repertoire;
(i) selecting at least 10 but no more than 30 antibody clones that are most frequent in the BCR repertoire and calculating a total frequency of the 10 to 30 frequent antibody clones;
(j) determining a variable region gene (V region) usage frequency in the antibody clones, optionally wherein the V is selected from the group consisting of IGHV4-30-2 heavy chain V region gene, IGHV4-30-4 heavy chain V region gene, IGHV3-23 heavy chain V region gene, IGHV4-34 heavy chain V region gene, and IGHV4-31 heavy chain V region gene;
(k) measuring a percent germline identity by comparing the V or J region in the BCR repertoire against a corresponding germline sequence; and
(l) determining the somatic mutation frequency in different regions along the V region.

59. The method of claim 57 or 58, further comprising the step of treating the patient with IgG-RT when the patient is selected for IgG-RT.

60. A method of treating a hypogammaglobulinemia patient, comprising administering immunoglobulin replacement therapy (IgG-RT) to the patient, wherein the patient has been selected for IgG-RT using the method of claim 57 or 58.
